(19) United States
(12) Patent Application Publication
(10) Pub. No.: US 2009/0197265 A1
(43) Pub. Date: Aug. 6, 2009

(54) EPIGENETIC ANALYSES

(75) Inventors: Bryan M. Turner, Birmingham (GB); Laura P. O'Neill, Birmingham (GB)

Correspondence Address: FULBRIGHT AND JAWORSKI LLP, 555 S. FLOWER STREET, 41ST FLOOR, LOS ANGELES, CA, 90071, US

(73) Assignee: THE UNIVERSITY OF BIRMINGHAM , a university

(21) Appl. No.: 12/162,194

(22) PCT Filed: Jan. 25, 2007
(86) PCT No.: PCT/GB2007/000243

§ 371 Date: Dec. 18, 2008

(30) Foreign Application Priority Data
Jan. 26, 2006 (GB) 0601538.2

(51) Int. Cl. C12Q 1/68

(52) U.S. Cl. 435/6

(57) ABSTRACT

A method of carrying out epigenetic analysis on an analyte biological sample, the method comprising carrying out chromatin immunoprecipitation on an analyte biological sample, characterised in that the method comprises a step of contacting the analyte biological sample with carrier chromatin. The method of the invention wherein the analyte biological sample comprises less than one million cells. The method of the invention wherein the analyte biological sample comprises less than about 6 μg of DNA and/or less than about 12 μg of chromatin.

The present invention relates to epigenetic analyses, and particularly although not exclusively, to methods for analysing epigenetic marks (or markers) in samples containing small cell numbers. In particular, the invention relates to a method for carrying out epigenetic comparison analyses of inner cell mass and embryonic stem cells using a novel and highly sensitive chromatin immunoprecipitation technique.

Epigenetics is the study of mechanisms by which the activity (expression) of genes is regulated. The changes in expression and silencing are caused by changes to the state of the DNA, which cause heritable changes in function without a change in the nucleotide sequence. Many genes in the body are permanently turned off (silenced) as part of normal development. But sometimes that process goes awry, turning off genes that should otherwise remain active, and vice versa. Hence, epigenetics, which is a field of study, and its associated therapies, aim to correct such defects by switching these genes back on, or off, as the case may be. This provides a new approach to the treatment of diseases, such as aging, inherited diseases and cancer.

Cultured embryonic stem cells (ES cells) are derived from the inner cell mass (ICM) of the pre-implantation blastocyst (early embryo). They were first derived in the mouse and have provided an invaluable resource for studies of differentiation and development. Like the ICM itself, ES cells are pluripotent, not only providing the starting material for transgenic mouse lines, but able to differentiate in culture to form embryoid bodies containing multiple cell lineages. The pluripotent state that characterises both ICM and ES cells is thought to be maintained by a relatively small number of crucial regulatory genes. Expression of two such regulators, Nanog and Oct4, both encoding homeodomain proteins, is necessary for pluripotency. Their silencing induces differentiation, while ES cells over-expressing Nanog continue to self-renew, even in media lacking growth factors previously thought to be essential to block differentiation.

A recent comprehensive analysis by Chromatin ImmunoPrecipitation (described below) and DNA microarrays (ChIP-Chip), of the binding of NANOG, OCT4 and the associated factor SOX2 to promoter regions in human ES cells, has shown that these proteins bind, often coordinately, to several hundred genes and that these genes may be transcriptionally active or silent. It seems that these key regulators both activate genes required for pluripotency (including themselves) and suppress genes that drive differentiation.

The programmes of gene activation and silencing that maintain pluripotency or drive cells down specific differentiation pathways involve multiple epigenetic changes beyond the binding of transcription factors, i.e changes to the state of the DNA, which cause heritable changes in function without a change in the nucleotide sequence. These changes include DNA methylation, chromatin condensation, replication timing and position within the nucleus. Central to these changes, and possible causative for some, is the enzyme-catalyzed, post-translational modification of the histones of the nucleosome core particle. Studies in many different systems have shown that specific modifications are often associated with specific functional states of chromatin. Increased acetylation (usually involving several or all four core histones) is a consistent marker of increased transcription, or of the potential for transcription, while histone lysine methylation is associated with either transcriptional activation or silencing, depending not only on which lysine residue is involved, but also on the degree of methylation, i.e. whether 1, 2 or 3 methyl groups are attached to the lysine ε-amino group. In at least some cases, the modification influences the transcriptional state through selective binding of a non-histone protein.

The histone modifications that are associated with expression or silencing of crucial genes during ES cell differentiation have been extensively studied by chromatin immunoprecipitation, CHIP, which is a technique used for analysing the association of specific proteins, or their modified isoforms, with defined genomic regions. A review of existing ChIP technology and the methodology conventionally used is found in O'Neill, L. P. & Turner, B. M. (2003) “Immunoprecipitation of native chromatin, NChIP”, Methods: A Companion to Methods in Enzymology 31, 76-82). The purpose of CHIP is to determine whether proteins including (but not limited to) transcription factors and modified histones, bind to a particular region on the endogenous chromatin of living cells or tissues. The in vivo nature of this technique is in stark contrast to other approaches traditionally used to study DNA-protein interactions by in vitro assays.

The principle underpinning ChIP is that fragments of the DNA-protein complex that packages the DNA in living cells (i.e. the chromatin), can be prepared in such a way (as described hereinafter) so as to retain the specific DNA-protein interactions that characterise each living cell. These chromatin (i.e. protein-DNA) fragments can then be immunoprecipitated using an antibody against the protein in question. The isolated chromatin fraction can then be treated to separate the DNA and protein components, and the identity of the DNA fragments isolated in connection with a particular protein (ie. the protein against which the antibody used for immunoprecipitation was directed), can then be determined by Polymerase Chain Reaction (PCR) or other technologies used for identification of DNA fragments of defined sequence.

Hence, chromatin immunoprecipitation essentially involves the following three key steps:—(i) isolation of chromatin to be analysed from cells; (ii) immunoprecipitation of chromatin using an antibody; and (iii) DNA analysis. The ChIP technique has two major variants that differ primarily in how the starting (input) chromatin is prepared. The first variant (designated NChIP) uses native chromatin prepared by micrococcal nuclease digestion of cell nuclei by standard procedures. However, problems associated with NChIP are that it is not usually useful for analysing non-histone proteins; selective nuclease digestion may bias input chromatin; and that nucleosomes may rearrange during digestion.

The second variant (designated XChIP) uses chromatin cross-linked by addition of formaldehyde to growing cells, prior to fragmentation of chromatin, usually by sonication. Some workers have used mild formaldehyde cross-linking followed by nuclease digestion, and UV irradiation has been successfully employed as an alternative cross-linking technique. However, problems associated with XChIP are that it is often extremely inefficient, cross-linking may fix (and thereby amplify) transient interactions between proteins and genomic DNA, and also that antibody specificity may be compromised by chemical changes in the protein that it recognises, induced by the cross-linking procedure, that are often not testable.

Furthermore, a major problem with either variant of ChIP is that it requires at least a million cells to be able to generate sufficient quantities of chromatin for the technique to work. Such a high number of cells is achievable with cultured cells, but is impossible with material from sources of low numbers of cells, for example, the early embryo, with a typical ICM comprising less than 60 cells (human) or 20 cells (mouse). For this key reason, it has not been possible, to date, to conduct epigenetic analyses, which study histone modifications, non-histone protein modifications, or DNA methylation associated with the silencing and activation of crucial regulatory genes, in samples where the number of cells is too low for ChIP to be effective, e.g. in the ICM or in other cell types in the early embryo. Because of this, it has also not been possible to address directly the crucial question of whether cultured ES cells provide a good model for the epigenetic mechanisms that drive differentiation in vivo.

Mager and Bartolomei (Nature Genetics, 2005, 37, 1194-1200) discuss various strategies for dissecting epigenetic mechanisms in the mouse. They state that, “although current protocols enable ChIP to be carried out on modest numbers of cells (>1×10⁶), many epigenetic processes occur during early development when only a few cells comprise a single embryo, currently ruling out “ChIP as a reasonable approach”.

It is therefore an object of the present invention to obviate or mitigate one or more of the problems of the prior art, whether identified herein or elsewhere, and to provide an improved method for carrying out epigenetic analysis in small cell numbers using chromatin immunoprecipitation (ChIP).

The inventors appreciated that chromatin immunoprecipitation (ChIP) has been used to study DNA modifications that are associated with expression or silencing of genes. However, at present, because such studies require such large numbers of cells, it is currently impossible to successfully use chromatin immunoprecipitation techniques in epigenetic analyses using genetic material from sources of lower numbers of cells, i.e. less than about one million cells. The inventors therefore set out to address this problem, in attempt to devise an improved method for epigenetic analysis having an improved sensitivity, and which could therefore be used with much smaller cell numbers than is currently possible.

Therefore, according to a first aspect of the present invention, there is provided a method of carrying out epigenetic analysis on an analyte biological sample, the method comprising carrying out chromatin immunoprecipitation on an analyte biological sample, characterised in that the method comprises a step of contacting the analyte biological sample with carrier chromatin.

The inventors have successfully devised a novel method of epigenetic analysis based on the use of chromatin immunoprecipitation (ChIP), which is applicable to very small amounts of biological material. The inventors have surprisingly demonstrated that as few as 10-15 analyte cells are required for the method according to the invention to work; however, preferably around at least 50 analyte cells are used. Hence, the method according to the invention allows epigenetic analyses, which study histone modifications, non-histone protein modifications, or DNA methylation associated with the silencing and activation of crucial regulatory genes, in samples where the number of cells is too low for normal chromatin immunoprecipitation to be effective, e.g. in the inner cell mass (ICM), or in other cell types in the early embryo. Furthermore, the inventors have found that cultured embryonic Stem (ES) cells do provide a good model for the epigenetic mechanisms that drive differentiation in vivo.

Advantageously, and surprisingly, the step of contacting the analyte biological sample with carrier chromatin greatly improves the sensitivity of the method according to the invention by at least four orders of magnitude compared to using standard chromatin immunoprecipitation in the absence of carrier chromatin. The inventors were incredibly surprised at this huge increase in sensitivity over existing ChIP techniques, and believe that this effect could not have been predicated in view of the prior art.

The term “epigenetic” will be known to the skilled technician, but for the sake of clarity, refers herein to the state or condition of DNA with respect to heritable changes in function without a change in the nucleotide sequence. Such changes are referred to in the art as epigenetic marks (or markers), and tend to result in expression or silencing of genes. Examples of epigenetic changes or marks, which may be caused by modification of DNA in the sample, or of proteins associated with it, and which may be analysed using the method according to the invention include histone protein modification, non-histone protein modification, and DNA methylation.

Hence, the term “epigenetic analysis” refers to determining the state, or condition of DNA, and its interaction with specific proteins and their modified isoforms in the analyte sample, and involves analysing or detecting epigenetic marks in the analyte biological sample. Hence, the method of the first aspect preferably comprises analysing or one or more detecting epigenetic marks in the analyte biological sample. It can be appreciated that the method of the invention may be used to assay epigenetic marks of any sort, on any gene, or region of the genome in the stem cells or stem cell precursor, or any other suitable analyte biological sample as discussed further below. As shown in the accompanying Examples, the inventors have used the method of the invention to investigate epigenetic marks present in regulator genes of stem cells, i.e. the Nanog; Oct4; and Cdx2 genes.

The term “chromatin immunoprecipitation” will also be known to the skilled technician, and preferably, comprises the following three steps:—(i) isolation of chromatin to be analysed from cells; (ii) immunoprecipitation of the chromatin using an antibody; and (iii) DNA analysis. It is therefore preferred that the analyte biological sample, which is subjected to chromatin immunoprecipitation, comprises chromatin. Chromatin is the substance of a chromosome and consists of a complex of DNA and protein (primarily histone) in eukaryotic cells, and is the carrier of the genes in inheritance. Chromatin occurs in two states, euchromatin and heterochromatin, with different staining properties, and during cell division it coils and folds to form the metaphase chromosomes. Hence, preferably, the analyte biological sample comprises nucleic acid, and preferably DNA, and any associated proteins.

Preferably, the chromatin under analysis is obtainable from at least one cell. Hence, preferably, the analyte biological sample comprises at least one cell. The cell may be derived from a tissue sample. Examples of cells that can be used as analyze biological samples for the method of the invention include the inner cell mass (ICM) of a blastocyst, for example a mouse blastocyst; embryonic stem cells (ES cells), for example cultured mouse ES cells; cells derived from tissue biopsies and sections, for example from cancerous tissue sections, including formaldehyde-fixed or ethanol-fixed tissue sections. Preferably the analyte biological sample comprises mammalian cells, preferably human or mouse cells.

As mentioned above, it is currently only possible to conduct epigenetic analyses using CHIP with very large cell numbers, i.e. at least one million cells, and usually 10 times more. However, advantageously, using the method of the first aspect, it is possible to successfully carry out epigenetic analyses on much smaller numbers of cells.

Hence, suitably, the method comprises carrying out chromatin immunoprecipitation using less than one million cells as the analyte biological sample, more suitably, less than 900,000 cells, even more suitably, less than 800,000 cells, still more suitably, less than 700,000 cells, and yet still more suitably, less than 600,000 cells. The inventors did not envisage it would be possible to carry out epigenetic analyses with such small cell numbers. Preferably, the method comprises carrying out chromatin immunoprecipitation on less than 500,000 cells, more preferably, less than 400,000 cells, even more preferably, less than 300,000 cells, still more preferably, less than 200,000 cells, and yet still more preferably, less than 100,000 cells. The inventors did not envisage it would be possible to carry out epigenetic analyses with such small cell numbers. It will be appreciated that such low numbers of cells is already several orders of magnitude less than the current state of the art, and therefore surprised the inventors.

However, in more preferred embodiments of the invention, the method comprises carrying out chromatin immunoprecipitation on less than 50,000 cells, more preferably, less than 25,000 cells, less than 20,000 cells, less than 15,000 cells, less than 10,000 cells, less than 5,000 cells, or 4,000 cells, or 3,000 cells or 2,000 cells, even more preferably, less than 1000 cells, still more preferably, less than 500 cells, and yet still more preferably, less than 250 cells. Surprisingly, and most preferably, the method comprises carrying out chromatin immunoprecipitation on less than 150 cells, more suitably, less than 100 cells, even more preferably, less than 75 cells, still more preferably, less than 40 cells, and yet still more preferably, less than 20 cells. As mentioned above, current use of chromatin immunoprecipitation in epigenetic analyses requires a minimum of at least a million cells and usually much more, thereby restricting its experimental or diagnostic use to cultured cell models or to situations where only large numbers of cells (i.e. at least a million cells) are available. Hence, the fact that the inventors of the present invention have demonstrated that as few as 10-20 cells is sufficient in their method was very surprising. The method of the invention is therefore useful for analysing the ICM or ES cells, which involve very low cell numbers. However, it is preferable that the method comprises carrying out chromatin immunoprecipitation using around at least 50 cells in the analyte biological sample.

It is estimated that one cell contains about 6×10⁻³ ng DNA per cell and equal amounts of DNA and protein in chromatin. Therefore, preferably, the method according to the invention comprises carrying out ChIP on less than about 6 μg DNA, or about 12 μg chromatin (this equates to mass of DNA or chromatin in about 1 million cells), and so on. The skilled technician will be able to calculate how much DNA and chromatin is contained within the preferred numbers of cell number given above, e.g. 3 μg DNA, or about 6 μg chromatin equates to 500,000 cells; 1.5 μg, or 3 μg chromatin equates to 250,000 cells; and 120 pg, or 240 pg chromatin equates to 20 cells, and so on.

The analyte biological sample is subjected to chromatin immunoprecipitation (ChIP). The skilled technician will appreciate that there are various types of ChIP techniques available depending on the type analyses to be conducted, as will be discussed hereinafter. Hence, a preferred ChIP technique used in accordance with the method of the invention may be NChIP or XChIP. The accompanying Examples provide detailed instructions as to how to perform ChIP. However, in summary, the method comprises a step of isolating chromatin from the biological sample. Cells may first be harvested using standard techniques, from which nuclei may then be obtained. For example, the cells may be disrupted (e.g. sonication), which results in the nuclei being released therefrom.

Following release of the nuclei, the method preferably comprises a step of digesting the nuclei in order to release the chromatin therefrom. In embodiments, where the method comprises use of NChIP, the chromatin is preferably isolated using nuclease digestion of cell nuclei by standard procedures. Preferably, micrococcal nuclease is added in the digestion. In embodiments, where the method comprises use of XChIP, the method may comprise a step of cross-linking the chromatin. This may be achieved for any suitable means, for example, by addition of a suitable cross-linking agent, such as formaldehyde, preferably prior to fragmentation of the chromatin. Fragmentation may be carried out by sonication. However, formaldehyde may be added after fragmentation, and then followed by nuclease digestion. Alternatively, UV irradiation may be employed as an alternative cross-linking technique.

The result of the step of isolating the chromatin is that proteins will become immobilised thereon. Once the proteins have been immobilized on the chromatin, the whole protein-DNA complex may then be immunoprecipitated. Hence, once the chromatin has been isolated, the method preferably comprises a step of immunoprecipitating the chromatin. Suitable techniques for the immunoprecipitation step will also be known to skilled technician, and the Examples describe a method for how this may be achieved. However, preferably immunoprecipitation is carried out upon addition of a suitable antibody against the protein in question. It will be appreciated that the suitable antibody will depend on what type of epigenetic analysis is being carried out (i.e. which mark is being analyzed).

Epigenetic analysis is the study of various changes (known as epigenetic marks) to the DNA of a cell, which tend to result in expression or silencing of genes. It should be appreciated that the method according to the invention may be used to assay epigenetic marks of any sort, on any gene, or region of the genome in the analyte. Examples of epigenetic marks, which may be caused by modification of DNA in the sample include histone protein modification, non-histone protein modification, and DNA methylation. Hence, it is preferred that the method according to the invention may comprise analysis or detection of histone protein modification, non-histone protein modification, and DNA methylation, or patterns thereof, in the biological sample.

Accordingly, the antibody used in the immunoprecipitation step may be immunospecific for non-histone proteins such as transcription factors, or other DNA-binding proteins. Alternatively, the antibody may be immunospecific for any of the histones H1, H2A, H2B, H3 and H4 and their various post-translationally modified isoforms and variants (eg. H2AZ). Alternatively, the antibody may be immunospecific for enzymes involved in modification of chromatin, such as histone acetylases or deacetylases, or DNA methyltransferases. Furthermore, it will be appreciated that histones may be post-translationally modified in vivo, by defined enzymes, for example, by acetylation, methylation, phosphorylation, ADP-ribosylation, sumoylation and ubiquitination of: defined amino acid residues. Hence, the antibody may be immunospecific for any of these post-translational modifications.

The accompanying Examples provide details of most preferred antibodies which may be used to achieve immunoprecipitation, i.e. antibodies with immunospecificity to H4K16ac (R252), H3K4me1 (R204), H3K4me2 (R148), H3K4me3 (R183). H3K9me2 and H4K8ac. Preferably, the chromatin is immunoprecipitated upon mixing with polyclonal antisera (serum containing antibodies) to the above histone modifications.

Following the immunoprecipitation step, the method preferably comprises a step of purifying DNA from the isolated protein/DNA fraction. This may be achieved by the standard technique of phenol-chloroform extraction.

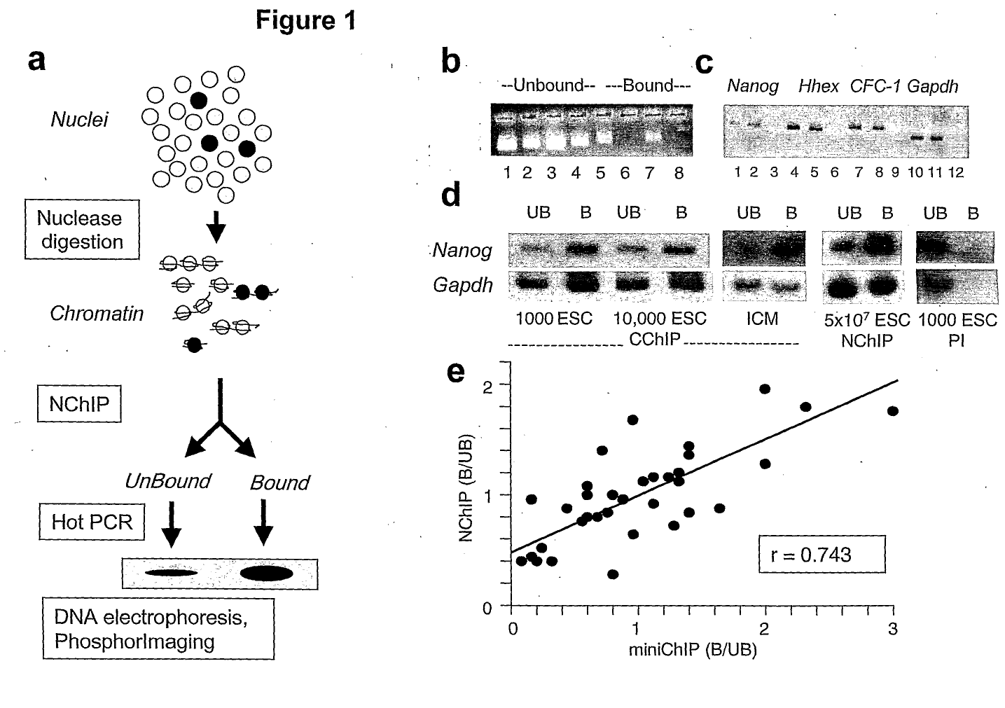

Following the purification step, the DNA fragments isolated in connection with the protein may then be analysed, and their identity determined. This is preferably achieved by PCR. For example, the analysis step may comprise use of suitable primers, which during PCR, will result in the amplification of a length of nucleic acid. The skilled technician will appreciate that the method according to the invention may be applied to analyse epigenetic marks on any genes or any region of the genome for which specific PCR primers may be prepared. However, by way of example only, preferred genes, which may be analysed include Nanog, Oct4 and Cdx2. Hence, preferred primers for amplifying regions of these genes are listed in Table 1 of the accompanying Examples. The PCR results may be viewed on an electrophoretic gel under suitable staining. Differences in sizes of bands may then be viewed and compared. The Examples and FIGS. 1-4 shows exemplary data.

By “suitable primers” the skilled person would understand that the chosen primers can be used for species-specific PCR, i.e. the primers can be used in a PCR that results in the amplification of a length of nucleic acid only from the analyte biological sample, but not from the carrier chromatin. Further information regarding the design of suitable primers is provided in the accompanying examples.

It will be appreciated that the method according to the invention comprises a step of contacting the analyte biological sample with carrier chromatin. The term “carrier chromatin” is used to define chromatin, which is adapted to act as a bulking agent. A further discussion on carrier chromatin that can be used in the invention is provided below. By mixing the carrier chromatin with the analyte chromatin, an analyte/carrier hybrid suspension is preferably produced.

The analyte biological sample (and preferably, chromatin thereof) may be contacted with the carrier chromatin after the step of isolating the nuclei from the sample, or even after the step of digesting the nuclei. However, it is most preferred that the step of contacting the analyte biological sample with the carrier chromatin is carried out at the beginning of the method of the invention, and preferably, before the step of isolating the nuclei from the biological sample, i.e. the step of contacting the analyte biological sample with carrier chromatin is performed before the step of carrying out chromatin immunoprecipitation on the analyte biological sample. Hence, the first step of the method comprises mixing analyte cells with carrier cells, and preferably an excess thereof. Hence, the carrier cells act as a bulking agent to enable ChIP to be carried out on very small numbers of analyte cells, i.e. less than 100, and preferably even less than 80 cells.

Advantageously, mixing the carrier chromatin with the chromatin under analysis, ensures that procedures necessary for chromatin preparation, i.e. nuclease digestion, or cross-linking and sonication, can be carried out in manageable volumes. For example, one million cells would typically be processed in a volume of 1 ml. To keep conditions comparable, 1000 cells would require a volume of 1 microlitre, feasible only with specially adapted technology, and 100 cells would require 0.1 μl. Alternatively, to use smaller cell numbers in a larger volume would result in a loss of material through surface attachment and handling that would substantially compromise recovery and invalidate the technique. Thus, the carrier chromatin acts as a bulking agent that protects the analyte sample from losses during the several steps that comprise the ChIP procedure. Preferably, the contacting step comprises mixing the carrier chromatin with chromatin derived from the analyte biological sample.

It is preferred that the carrier chromatin is obtained from a plurality of cells, referred to herein as carrier cells. It is therefore preferred that the method comprises mixing cells of the analyte biological sample with a large excess of carrier cells. Hence, the method according to the invention differs from existing ChIP methods because the analyte cells are first mixed with a large excess of a carrier cell population. Suitably, the excess of carrier chromatin/cells (calculated as either mass of chromatin or number of cells) should be at least 10 times the amount of analyte chromatin/cells. More suitably, the excess of carrier chromatin/cells should be at least 100 times, even more suitably, at least 500 times, and even more suitably, at least 1000 times the amount of analyte chromatin/cells. However, it is preferred that the excess of carrier chromatin/cells should be at least 10,000 times, even more suitably, at least 100,000 times, and even more suitably, at least 1,000,000 times the amount of analyte chromatin/cells. In practise, the inventors have found that between 15 and 10,000 analyte cells should be mixed with about 50 million carrier cells.

The inventors carefully considered various types of carrier chromatin in accordance with the invention. While the inventors do not wish to be bound by any hypothesis, they believe that there are two requirements, which contribute to the success of the method of the invention. Firstly, it is preferred that the analyte cells and the carrier cells have similar behaviours when subjected to homogenisation to prepare their corresponding nuclei and, subsequently, to micrococcal nuclease digestion to prepare chromatin therefrom. It is preferred that the method prepares chromatin from the carrier cells and the analyte with substantially equal efficiency. If this is not achieved, the inventors have found that the method is not as efficient. Secondly, it is a requirement that the DNA of the analyte chromatin may be distinguished from the carrier chromatin. Hence, preferably, the carrier chromatin (and hence, carrier cells) and the analyte chromatin (and hence, analyte cells) are sufficiently different, genetically from each other, such that a sequence (e.g. a gene) from the analyte cells may be assayed accurately and specifically by a suitable assay, such the Polymerase Chain Reaction (PCR), even in the presence of a large excess (e.g. up to six orders of magnitude) of carrier DNA. For example, according to taxonomic classification organisms, the carrier chromatin and analyte biological sample may be derived from different species, different genera, different families, different orders, different classes, different phyla, or different kingdoms.

In the accompanying Examples, the inventors describe the effective use of the long-established cell line, Drosophila SL2, as suitable carrier, which has been demonstrated to meet both of the criteria above when the analyte biological sample is derived from a mouse, i.e. the carrier chromatin and analyte biological sample are derived from organisms from different phyla. However, the inventors believe that many other cell types could also be used meeting both these criteria. Therefore, it is preferred that the carrier chromatin is obtainable from any suitable eukaryotic or prokaryotic cell, although the choice is limited by the key requirements of comparable behaviour during isolation of nuclei and chromatin (see above) and distinctiveness of DNA sequence to allow discrimination of isolated DNA by PCR. An especially preferred source for the carrier chromatin is Drosophila, and particularly the Drosophila SL2 cell line. That is, where the analyte biological sample is not derived from Drosophila then the carrier chromatin is derived from Drosophila cells, preferably Drosophila SL2 cells. Other suitable sources of carrier chromatin may be identified e.g. plant cells, such as those derived from Arabidopsis.

The inventors believe that there are a number of valid reasons why the use of carrier chromatin in the method according to the invention may not work, and which therefore support the argument that the method of the invention is not obvious. For example, the inventors expected that the chromatin in the analyte-carrier hybrid suspension would not be digested in the presence of excess carrier. They also expected that the antibodies would not bind to the chromatin in the analyte-cell hybrid suspension in the presence of excess carrier. Furthermore, the inventors expected that the PCR reaction would not be able to detect analyte cell DNA in the presence of excess carrier chromatin. Hence, the inventors believe that it was by no means obvious that mixing the analyte nucleic acid with carrier chromatin would work, and it is very surprising that it does, with such efficacy.

Accordingly, in a preferred embodiment, the method comprises a series of steps including: a step of mixing carrier cells with analyte cells; a step of disrupting the cells to release nuclei therefrom; a step of digesting the nuclei to release the chromatin therefrom; a step of immunoprecipitating the chromatin; a step of purifying DNA from the isolated protein/DNA fraction; and step of analysing DNA fragments isolated in connection with the protein.

As mentioned herein, epigenetic analyses is the study of various changes (known as epigenetic marks) to the DNA of a cell, which tend to result in expression or silencing of genes. Examples of epigenetic marks, which may be analyses using the method according to the first aspect include histone protein modification, non-histone protein modification, and DNA methylation.

Hence, according to a second aspect of the invention, there is provided a method of identifying histone protein modification, non-histone protein modification, and/or DNA methylation, or patterns thereof, in an analyte biological sample, the method comprising carrying out chromatin immunoprecipitation on an analyte biological sample, characterised in that the method comprises a step of contacting the analyte biological sample with carrier chromatin.

It should be appreciated that the method according to the second aspect may be used to assay epigenetic marks of any sort, on any gene, or region of the genome in the analyte, which comprise histone protein modification, non-histone protein modification, and/or DNA methylation, or patterns thereof. The method according to the second aspect is therefore useful for analysis of the distribution of histone modifications and variants thereof. The four types of histone protein include: H1 (‘lysine-rich’), H2A and H2B (‘slightly lysine rich’), and H3 and H4 (‘arginine-rich’), any of which may be analysed using the method of the invention.

The chromatin immunoprecipitation step in the method according to the second aspect may therefore comprise use of a suitable antibody, which may be directed against non-histone proteins, such as transcription factors or other DNA-binding proteins. In order to apply the method of the invention to the analysis of non-histone proteins, preferably cells (or nuclei therefrom) are treated with a cross-linking reagent, typically formaldehyde. However, other cross-linking agents may be used, such as glutaraldehyde or UV irradiation. This prevents the proteins dissociating from the DNA during chromatin preparation.

Alternatively, the immunoprecipitation step may comprise use of a suitable antibody, which may be directed to any of the histones: H1, H2A, H2B, H3 and H4 and their various post-translationally modified isoforms and variants (eg. H2AZ). Alternatively, the immunoprecipitation step may comprise use of a suitable antibody, which may be directed enzymes involved in modification of chromatin, such as histone acetylases or deacetylases, or DNA methyltransferases.

As described above, histones may be post-translationally modified in vivo, by defined enzymes, by acetylation, methylation, phosphorylation, ADP-ribosylation, sumoylation and ubiquitination of defined amino acid residues, and all these modifications are amenable to study by use of antibodies of the appropriate secificity. Hence, the method according to the second aspect may be used to identify patterns or levels of protein acetylation, protein methylation, protein phosphorylation, protein ADP-ribosylation, protein sumoylation or protein ubiquitination (i.e. histone or non-histone protein modifications). Preferably, the method according to the second aspect may be used to identify patterns, distribution, or levels of DNA methylation (5-methyl cytosine) associated with individual genes. In order to do this, DNA fragments are preferably prepared from the digested chromatin by standard procedures, prior to incubation with an anti-5meC antibody.

Increased acetylation (usually involving several or all four core histones) is a consistent marker of increased transcription, or of the potential for transcription, while histone lysine methylation is associated with either transcriptional activation or silencing, depending not only on which lysine residue is involved, but also on the degree of methylation, i.e. whether 1, 2 or 3 methyl groups are attached to the lysine ε-amino group. Therefore, preferred histone modifications, which may be analysed with the method of the invention, include H4K16ac; H3K4me3; H3K9me2; H3K4me1; and H4K8ac, as described in the accompanying Examples.

The inventors believe that the method according to the first or second aspects have many applications, and may be applied to a wide variety of analyte biological sample types. Furthermore, the inventors have appreciated that each of the variables (histone protein modification, non-histone protein modification, and/or DNA methylation) are key regulators of gene expression, and changes in them are associated with altered cell function or disfunction, and hence disease. It is therefore likely that epigenetic markers will become increasingly valuable diagnostic and prognostic indicators and guides to appropriate treatment regimens.

Hence, in a third aspect, there is provided use of the method according to the first or second aspect for the diagnosis or prognosis of a disease condition.

It should be appreciated that the method according to the third aspect may be used to assay epigenetic marks of any sort, on any gene, or region of the genome in the analyte. Examples of disease which may be diagnosed, or for which a prognosis may be established using the method according to the first or second aspect, include all types of cancer, such as prostate, cervical cancer, or Hodgkin's lymphoma, and autoimmune diseases, such as rheumatoid arthritis. Preferably, the diagnostic method is carried out in vitro.

For example, the method of the third aspect may comprise taking first and second samples, mixing same with carrier cells (chromatin), and carrying out ChIP as defined herein to enable an epigenetic comparison thereon. For example, the first sample may comprise normal (a control) cells, and the second sample may comprise diseased cells, and in which the epigenetic marks may be compared using the method according to the first or second aspects. By epigenetic comparison, the method of the invention can be used to categorise an analyte biological sample as being diseased or non-diseased.

The samples may be obtained from healthy and diseased tissues. The biological sample may comprise material from tumours and adjacent normal tissue taken during surgery or by needle biopsy. Samples from solid tissue or tumours may require treatment prior to mixing with the carrier cells to produce a cell suspension suitable for mixing with the carrier chromatin. For example, said treatment may comprise mild digestion with proteolytic enzymes to break cell-cell contacts. The carrier cells may then be added thereto, followed by ChIP.

Hence, the method according to the third aspect preferably comprises taking a sample of cells from a tissue under investigation, and a control tissue. An example of a tissue under investigation includes bone marrow. The method comprises carrying out the method according to the first aspect or second aspect, and then analysing the sample, prior to the use of the main sample for transplantation. Analyte cells may be derived from “buffy coat” material, ie. surface layer cells after centrifugation (a standard clinical laboratory technique); or after further purification of selected cells by antibody labelling and chromatography (e.g. immunoaffinity purifications).

The biological sample may also comprise cells of defined types (lineages) prepared by flow cytometry; laser dissection microscopy; differential centrifugation; or other standard fractionation techniques. The accompanying examples set out how a population of analyte sample cells can be prepared using fluorescent-activated cell sorting or from tissue samples.

The method of the invention may also be used to analyse cells from ethanol-fixed or formaldehyde-fixed tissue sections, which would allow epigenetic analysis of archived sections of normal and pathological tissue, for example cervical cancer.

The inventors have appreciated that it is currently impossible to use chromatin immunoprecipitation in epigenetic analyses using material from the early embryo itself, because a typical Inner Cell Mass (ICM) comprises less than 20 cells, i.e. far below the current ChIP requirement of at least one million cells. Furthermore, it has also not been possible to address directly the crucial question of whether cultured ES cells provide a good model for the epigenetic mechanisms that drive differentiation in vivo. The inventors therefore carried out investigations to see if it was possible to make a quantitative comparison of the distribution of histone modifications on key regulator genes in ICM and cultured ES cells to answer these questions. Hence, the inventors investigated the regulator genes Nanog, Oct4 and Cdx2 in ICM and ES cells. The inventors demonstrate in the accompanying Examples that surprisingly it was possible make a quantitative comparison of the distribution of histone modifications on each of these regulator genes in the ICM and cultured ES cells, and that accordingly, cultured ES cells do provide a good model for the epigenetic mechanisms that drive differentiation in vivo.

Hence, in a fourth aspect there is provided a method of carrying out epigenetic analysis on a sample of stem cells or a stem cell precursor, the method comprising carrying out chromatin immunoprecipitation on a sample of stem cells or a stem cell precursor, characterised in that the method comprises a step of contacting the stem cells or stem cell precursor with carrier chromatin.

The skilled technician will appreciate that embryonic stem cells are derived from the inner cell mass (ICM), which is the inner component of a blastocyst. The ICM is surrounded by an outer trophectoderm (which consists of a trophoblast). Hence, the cells under analysis using the method according to the invention may be stem cells derived from any of these components. Accordingly, it is preferred that stem cell precursor may comprise an ICM, or any of the cells from the ICM, such as the trophectoderm or trophoblast.

Preferably, the method comprises taking a sample of stem cells or stem cell precursor from an individual, and then using the methods according to the invention to carry out epigenetic analysis thereon. The accompanying Examples provide details of how to carry out trophosurgery to obtain suitable analyte cells. It should be appreciated that the method according to the fourth aspect may be used to assay epigenetic marks of any sort, on any gene, or region of the genome in the stem cells or stem cell precursor. However, a preferred analysis comprises analysis of a regulator gene in the stem cells or precursor. Preferred regulator genes which may be analysed may be independently selected from: Nanog; Oct4; and Cdx2. It is more preferred that histone modification may be analysed on the gene of interest. Preferred histone modifications, which may be analysed include H4K16ac; H3K4me3; H3K9me2; and H3K4me1, as described in the accompanying Examples.

Preferably, the step of contacting the stem cells or stem cell precursor with carrier chromatin is the first step of the method, wherein stem cells or cells from a stem precursor are mixed with carrier cells, and preferably an excess thereof. Hence, the carrier cells act as a bulking agent to enable ChIP to be carrier out on very small numbers of stem cells or a precursor thereof, i.e. less than 100, and preferably even less than 80 cells. Preferred primers used in PCR analysis in the ChIP method are found in Table 1.

In summary, the inventors have devised a novel CHIP procedure (referred to herein as CChIP) based on the use of Drosophila carrier chromatin, which allows the definition and quantification of patterns of histone modification in fewer than 100 cells. The inventors have demonstrated that established activating and silencing epigenetic marks, such as H4 acetylation, H3K4 tri-methylation and H3K9 di-methylation, are similarly distributed across regulator genes such as Nanog and Oct4 in ICM and ES cells, while other modifications provide evidence for subtle epigenetic differences.

Hence, the first step for each of the methods according to the invention comprises a step of mixing the analyte cells with a large excess of carrier cells. The inventors believe that each of the methods according the invention represents an outstanding advance in the field of epigenetic analysis using chromatin immunoprecipitation due to the significant increase in sensitivity on small cell numbers (i.e. less than 10 million cells). The only alternative approach to chromatin immunoprecipitation (ChIP) analysis of small cell numbers instead of contacting the analyte sample with carrier chromatin would be to miniaturise each step of the method. Hence, to reduce the cell numbers required by even 1000 fold (i.e. in order to allow analysis of 50,000 cells rather than 50 million) would require a 1000-fold reduction in reaction volume. Accordingly, digestions and incubations currently carried out in 1 ml reaction volume would therefore have to be reduced to 1 μl reaction volume. It will be appreciated that a lab technician would not be able to work with these small volumes using standard lab equipment. Hence, it would require extensive investment in sophisticated measuring equipment and liquid handling technology. Furthermore, this would still result in the number of cells required in the reaction being more than three orders of magnitude above the 10-15 cells in the method according to the invention. Hence, miniaturising each step of the ChIP method would have significant disadvantages and would still be nowhere near as sensitive as the method according to the invention.

According to a fifth aspect of the present invention, there is provided an epigenetic analysis kit comprising one or more materials for performing chromatin immunoprecipitation, characterised in that the kit further comprises carrier chromatin.

As set out above, the use of carrier chromatin in the methods of the invention greatly improves the sensitivity of the methods of the invention such that chromatin immunoprecipitation (ChIP) analysis can be performed on very small analyte biological samples. The kit of this aspect of the invention includes a quantity of such carrier chromatin and can be employed when performing the methods of the invention. Since it has not previously been considered that carrier chromatin can greatly improve the sensitivity of epigenetic analysis using ChIP, the kit of this aspect of the invention has important advantages over exiting kits for performing ChIP.

The term “epigenetic analysis” is described further in the first aspect of the invention set out above. Thus the kit can be used to analyse or detect epigenetic marks in an analyte biological sample, such as histone protein modifications, non-histone protein modifications, and DNA methylation.

By “materials for performing chromatin immunoprecipitation” we include reagents required for performing a ChIP analysis on an analyte biological sample, and, optionally, one or more positive or negative controls. Reagents required when performing chromatin immunoprecipitation are set out in the accompanying examples and are included in this aspect of the invention. Such reagents include NB buffer (15 mM Tris-HCL pH 7.4, 60 mM KCl, 15 mM NaCl, 5 mM MgCl₂, 0.1 mM EGTA, 0.5 mM 2-mercaptoethanol, 0.1 mM PMSF); digestion buffer (50 mM Tris-HCl pH7.4, 0.32M sucrose, 4 mM MgCl₂, 1 mM CaCl₂, 0.1 mM PMSF); lysis buffer (2 mM Tris-HCl pH 7.4, 0.2 mM EDTA, 5 mM Na butyrate, 0.2 mM PMSF, 0.4 mM glycine). Other reagents, such as the fixation buffer used in X-ChIP are also provided in the accompanying examples and are included in this aspect of the invention.

As well as these materials, the kit may contain one or more antibodies suitable for the immunoprecipitation of commonly studied proteins. Where epigenetic analysis requires immunoprecipitation of chromatin using one or more antibodies to histones or their various post-translationally modified isoforms and variants, then the kit may also include one or more of the antibodies to histone set out above and in the accompanying examples, i.e. antibodies with immunospecificity to H4K16ac (R252), H3K4me1 (R204), H3K4me2 (R148), H3K4me3 (R183). H3K9me2 or H4K8ac.

The kit may also comprise one or more primers suitable for Polymerase Chain Reaction (PCR) analysis of specific regions of DNA. For example, as set out in the accompanying examples, the epigenetic analysis methods of the invention can be used to determine epigenetic marks associated with the regulator genes Nanog, Oct4 and Cdx2 in ICM and ES cells. Therefore, the kit may also comprise one or more of the primers or primer pairs set out in Example 1.

The term “carrier chromatin” is described further in the first aspect of the invention set out above. As will be appreciated, a requirement of the carrier chromatin is that it has similar properties to the chromatin present in the analyte biological sample when performing the methods of the invention, but that the DNA of the analyte chromatin may be distinguished from the carrier chromatin. Therefore the nature of the carrier chromatin necessarily varies according to the analyte biological sample to be studied with the kit of the invention. However, as set out in the accompanying examples, the inventors describe the effective use of the long-established cell line, Drosophila SL2, as suitable carrier, which has been demonstrated to meet both of the criteria above when the analyte biological sample is derived from a mouse, i.e. the carrier chromatin and analyte biological sample are derived from organisms from different phyla. Therefore, in a preferred embodiment of this aspect of the invention the carrier chromatin is from Drosophila SL2 cells. Alternative embodiments of the invention may include where the carrier chromatin is derived from a plant cell, such as Arabidopsis cells.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

FIG. 1 shows (A) Summary of the method according to the invention, i.e. the CChIP protocol; (B) Agarose gel electrophoresis of DNA from precipitated (Bound) and unprecipitated (Unbound) chromatin fractions from two typical CChIP experiments in which 107 SL2 cells were mixed with 1000 (tracks 1,2,5,6) or 10,000 (tracks 3,4,7,8) mouse ES cells. DNA in tracks 1,3,5 and 7 is from chromatin treated with anti-H4K8ac and that in tracks 2,4,6, and 8 is a no-antibody control; (C) Detection of specific mouse genes by hot PCR, electrophoresis and PhosphorImaging, in the antibody-bound fraction from a CChIP experiment with 1000 mouse ES cells and antibody to H4K8ac. Each amplified DNA sample was loaded in duplicate (tracks 1/2,4/5,7/8, 9/10) alongside a track containing Drosophila DNA alone (tracks 3,6,9,12). Data was collected only if PCR resulted, as shown, in radiolabelled bands of the appropriate size with no detectable amplification of Drosophila DNA; (D) Typical results from CChIP experiments with the indicated number of mouse ES cells (ESC), or three ICMs (˜50 cells) prepared by immunosurgery (ICM), compared with results from a conventional NChIP experiment using 5×10⁷ undifferentiated mouse ES cells. Material precipitated non-specifically in control tubes either without added antibody or with preimmune (PI) IgG did not give a detectable PCR signal; (E) Comparison between levels of histone modification (B/UB ratios) at specific gene regions assayed by conventional NChIP (starting with 5×10⁷ mouse ES cells) and CChIP, using 1000 mouse ES cells. Data is from 7 lineage-specific genes after precipitations with antibodies to five different histone modifications (H4K8ac, H3K4me1, H3K4me2, H3K4me3 and H3K9me2). To eliminate the possibility of a spurious correlation generated by the differing precipitation efficiencies of the five different antisera, individual B/UB ratios for each antibody were normalised by dividing by the mean B/UB ratio for that antibody. The least-squares regression line and Pearson product moment correlation coefficient (r) are indicated;

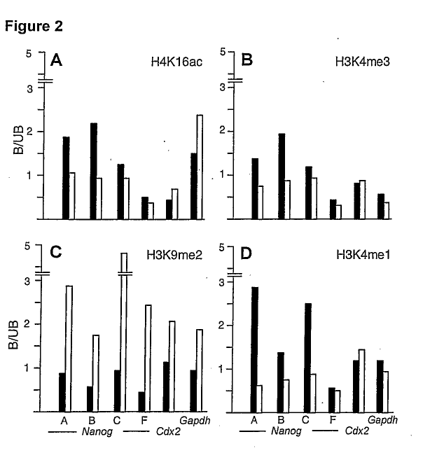

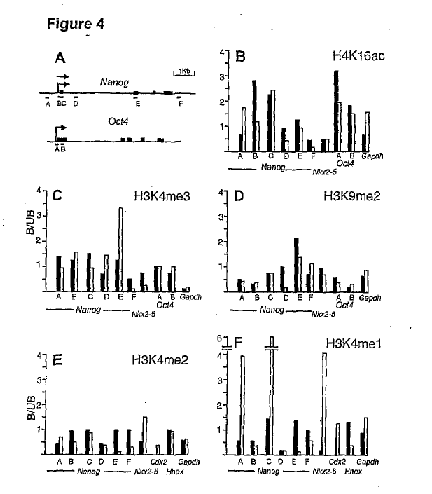

FIG. 2 shows quantitation by conventional NChIP of levels of histone modification (B/UB ratios) at selected regions of Nanog and the promoter regions of Cdx2 and Gapdh in undifferentiated ES cells (dark), or embryoid bodies formed after 7 days differentiation in culture (pale). Regions A, B, C and F of Nanog are as indicated in FIG. 4A. Immunoprecipitation was with antibodies to H4K16ac (A), H3K4me3 (B), H3K9me2 (C) and H3K4me1 (D), as indicated;

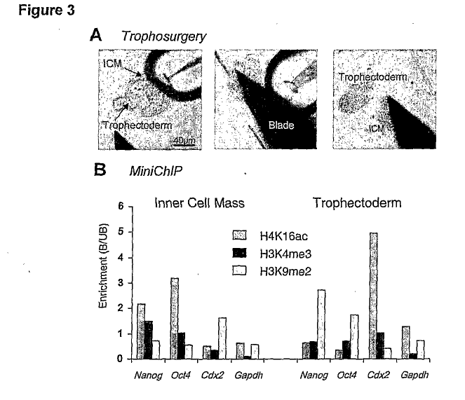

FIG. 3 shows (A) Separation of the ICM and trophectoderm by manual dissection. The hatching blastocyst was held by a suction pipette (top right) and part of the trophectoderm cut away so as to ensure no contamination with ICM. This was then used for CChIP. The ICM with residual trophectoderm still attached was discarded. ICM for CChIP, free of trophectoderm, was prepared by immunosurgery; (B) Quantitation by CChIP, of levels of histone modification (B/UB ratio) associated with the promoter regions of specific genes in the Inner Cell Mass and Trophectoderm;

FIG. 4 shows (A) Locations of primer pairs A-F on the Nanog gene and primer pairs A and B on Oct4; (B-F) Quantitation by CChIP of levels of histone modification (B/UB ratio) at selected regions of Nanog and the promoter regions of other genes in Inner Cell Mass (dark) and cultured ES cells (pale). Immunoprecipitation was with antibodies to H4K16ac (B), H3K4me3 (C), H3K9me2 (D), H3K4me2 (E) and H3K4me1 (F), as indicated.

### EXAMPLE 1

### Use of CChIP Analysis

The inventors appreciated that the method of chromatin immunoprecipitation (ChIP) has been used to study nucleic acid modifications (histone modification, non-histone modification, and DNA methylation) that are associated with expression or silencing of genes. However, because such studies require at least ten million cells, they realised that it is impossible to use chromatin immunoprecipitation using material from the early embryo itself, with a typical ICM comprising less than 20 cells. The inventors therefore set out to address this problem, and devised the method according to the invention having a greatly improved sensitivity, based on chromatin immunoprecipitation method (referred to herein as “CChIP”). The inventors also wanted to see if it was possible to make a quantitative comparison of the distribution of histone modifications on key regulator genes in ICM and cultured ES cells. Hence, the inventors investigated the regulators Nanog, Oct4 and Cdx2 in ICM and ES cells.

### Materials and Methods

### Cells

Drosophila SL2 cells were grown at 26° C. in Schneider's medium (Gibco) supplemented with 8% foetal calf serum (FCS, Gibco) and antibiotics (Penicillin/Streptomycin) (J. Embryol. Exp. Morphol. 27, 353-365 (1972)). Mouse ES cell line PGK12.1 was grown as previously described in monolayer culture, without feeder cells, in medium supplemented with the growth factor LIF (Cell 77, 41-51 (1994), EMBO J. 18, 2897-2907). Cells were induced to differentiate by replating on non-adherent plastic in the absence of LIF (Annu Rev Cell Dev Biol 17: 435-62 (2001)).

### Antibodies

Rabbit polyclonal antisera (serum containing antibodies) to H4K16ac (P252), H3K4me1 (R204), H3K4me2 (R148), H3K4me3 (R183) and H3K9me2 (Upstate.), were raised by immunization with synthetic peptides conjugated to ovalbumin as previously described (Eur. J. Biochem. 179, 131-139 (1989), Methods 19, 417-424 (1999)). Specificity was assayed by inhibition ELISA for all in-house and commercial antisera used (Genes Dev. 18, 1263-1271 (2004), Methods 19, 417-424 (1999)) and checked by western blotting. For all antisera, cross-reaction with epitopes other than that against which the antiserum was raised was insignificant.

### Trophosurgery

Cryopreserved 2-cell Balb/c mouse embryos (Embryotech, USA) were thawed and cultured in M16 media (Specialty Media) to the expanded blastocyst stage. Isolation of the inner cell mass (ICM) was achieved by immunosurgery using the method developed by Solter and Knowles (Proc. Natl. Acad. Sci. U.S.A. 72, 14967-14972 (1975)), with minor alterations. Briefly, the zona pellucida was removed from cultured blastocysts using 0.5% Pronase (Sigma) and exposed to 1:100 dilution of rabbit anti-mouse serum (Sigma). Blastocysts were washed with PBS and transferred to guinea pig complement (Sigma). Dead trophectoderm cells were removed and each ICM transferred into a single droplet of trypsin-EDTA solution and incubated to allow the cells to disaggregate.

For isolation of trophectodermal cells, a small area of the zona pellucida, at the opposing pole to the ICM, was dissolved by zona drilling with a micropipette (The Pipette Company, Ltd.), using acidified Tyrode's solution (Sigma). Trophectoderm (TE) isolation was achieved by a period of culture (up to 3 hours) after zona drilling, during which the expansion of the blastocoel cavity forces the TE to herniate out of the small opening in the zona. The herniating TE vesicle was sliced away from the ICM, retained in the zona pellucida, by micromanipulation with an ultra-sharp micro-splitting blade (AB Technology) at the herniated junction.

### Chromatin ImmunoPrecipitation

For the CChIP procedure, Drosophila SL2 cells were pelleted and washed 3× in ice cold PBS, 5 mM sodium butyrate. Cells were resuspended to 5×10⁷ cells/ml and 1 ml aliquots were mixed with a small number (usually 100-1000) of mouse cells (either ES cells, ICM or trophectoderm). Cell mixtures were washed twice in NB buffer (15 mM Tris-HCL pH 7.4, 60 mM KCl, 15 mM NaCl, 5 mM MgCl₂, 0.1 mM EGTA, 0.5 mM 2-mercaptoethanol, 0.1 mM PMSF) supplemented with 5 mM sodium butyrate. The final pellet was resuspended in 2 ml NB buffer, mixed with 2 ml 1% Tween 40 in NB buffer and stirred for one hour on ice. Nuclei were released by homogenisation in a Dounce all glass homogeniser with a “tight” pestle using four cycles of 10 strokes with pauses of 10-15 min (on ice) between each cycle to prevent excessive foaming. This should result in a 75-80% yield of intact nuclei. Nuclei were pelleted (2500 rpm, MSE 3000, 15 min. 4° C.), resuspended in 20 ml NB buffer, 5% (v/v) sucrose and pelleted (3000 rpm, MSE 3000, 25 min, 4° C.). Nuclei were resuspended in 5 ml digestion buffer (50 mM Tris-HCl pH7.4, 0.32M sucrose, 4 mM MgCl₂, 1 mM CaCl₂, 0.1 mM PMSF). A₂₆₀ was measured to give a rough estimate of DNA concentration and the sample adjusted to 250 ug/ml. 1 ml aliquots were mixed with 50 U micrococcal nuclease (Pharmacia) and incubated for 5 min at 28° C.

The digested samples were spun at 300 rpm, 5 min and the supernatant removed (fraction S1). The pellet was resuspended in 1 ml lysis buffer (2 mM Tris-HCl pH 7.4, 0.2 mM EDTA, 5 mM Na butyrate, 0.2 mM PMSF, 0.4 mM glycine) to give fraction S2. Both fractions were dialysed overnight at 4° C. against lysis buffer, centrifuged (3000 rpm, 10 min MSE 3000) and the supernatants (fractions S1 and S2) analysed by agarose gel electrophoresis. If the chromatin was satisfactory (ie. predominantly but not exclusively mononucleosomes with minimal sub-nucleosomal fragments), S1 and S2 were combined and used for chromatin immunoprecipitation (ChIP), which was carried out exactly as previously described (Hum. Mol. Genet. 12, 1783-1790 (2003), EMBO J. 14, 3946-3957 (1995)), with the exception that derived DNA fractions were analysed by PCR.

### Polymerase Chain Reaction

All PCR reactions were performed in duplicate with mouse and Drosophila DNA controls run in parallel to monitor cross-hybridization. 45 μl of Reddy Mix PCR Master Mix (AB Gene, UK) was added to 3 ul input DNA (about 25 ng) and 2 ul primer mix. 1 μl (1 μCi) of dCTP radiolabelled with α-³²P (Amersham, UK) was added to each PCR reaction before cycling. In the standard protocol, aliquots were removed after 38 and 41 cycles and loaded onto 5% polyacrylamide gels and electrophoresed at 400 volts and 30 mA for 15 minutes. Gels were dried onto filter paper (SpeedGel System, Thermo Savant, UK) for a minimum of 2 hours. Filters were exposed to a phosphor screen overnight and scanned with a PhosphorImager (Typhoon 9200, Amersham, UK). Intensity values for each PCR product were analysed with ‘Image Quant 5.2’ software (Molecular Dynamics). Primer pairs used are listed in Table 1.

TABLE 1
Primer pairs used in PCR

| Primer | Gene | | |
|---|---|---|---|
| Name | Name | Forward | Reverse |
| Primer 26 | Nanog | 5′-gtaaagcctctttttgggg-3′ | 5′-caccagccctgtgaatt-3′ |
| | | (SEQ ID No:1) | (SEQ ID No:2) |
| Primer A | Nanog | 5′-ctatcgccttgagccgttg-3′ | 5′-aactcagtgtctagaaggaaagatca-3′ |
| | | (SEQ ID No:3) | (SEQ ID No:4) |
| Primer | Nanog | 5′-tcacactgacatgagtgtgg-3′ | 5′-tctgtgcagagcatctcagt-3′ |
| 14.1 | | (SEQ ID No:5) | (SEQ ID No:6) |
| Primer B | Nanog | 5′-gtggagcgctaggaagtgtg-3′ | 5′-agccagggctacacagagaa-3′ |
| | | (SEQ ID No:7) | (SEQ ID No:8) |
| Primer 1 | Nanog | 5′-gaagacctgcctcttcaagg-3′ | 5′-agaacacagtccgcatctt-3′ |
| | | (SEQ ID No:9) | (SEQ ID No:10) |
| Primer D | Nanog | 5′-accagcccctggtttatttt-3′ | 5′-ggcaaagataagtgggcaga-3′ |
| | | (SEQ ID No:1l) | (SEQ ID No:12) |
| Primer 31 | Cdx2 | 5′-aaatcgtgtttctgggg-3′ | 5′-ccttacgtgattaacgagtg-3′ |
| | | (SEQ ID No:13) | (SEQ ID No:14) |
| Primer 11 | Cfc1 | 5′-tagggtcagcacttccag-3′ | 5′-cacagtcttgcaagatgaag-3′ |
| | | (SEQ ID No:15) | (SEQ ID No:16) |
| Primer R | Gapdh | 5′-tgtgccaagcacttgtataac-3′ | 5′-tatgtctgaccagaggagagca-3′ |
| | | (SEQ ID No:17) | (SEQ ID No:18) |
| Primer | Hhex | 5′-tcccccgttctagacagt-3′ | 5′-agcctctggaacctgga-3′ |
| L.1 | | (SEQ ID No:19) | (SEQ ID No:20) |
| Primer 3 | Oct4 | 5′-ctgtaaggacaggccgagag-3′ | 5′-caggaggccttcattttcaa-3′ |
| | | (SEQ ID No:21) | (SEQ ID No:22) |
| Primer | Oct4 | 5′-ggatggcatactgtggacct-3′ | 5′-agttgctttccactcgtgct-3′ |
| Exon1 | | (SEQ ID No:23) | (SEQ ID No:24) |
| Primer | Oct4 | 5′-caaggcaagggaggtagaca-3′ | 5′-gctcctgatcaacagcatca-3′ |
| Exon4 | | (SEQ ID No:25) | (SEQ ID No:26) |
| Primer 6 | Nkx2-5 | 5′-gatttcacacccaccctc-3′ | 5′-ccggtcctagtgtggaat-3′ |
| | | (SEQ ID No:27) | (SEQ ID No:28) |
| Primer 35 | Cfc1 | 5′-caaagctctttggtttgttg-3′ | 5′-agcctcctggtgctatttac-3′ |
| | | (SEQ ID No:29) | (SEQ ID No:30) |
| Primer 11 | Cfc1 | 5′-tagggtcagcacttccag-3′ | 5′-cacagtcttgcaagatgaag-3′ |
| | | (SEQ ID No:31) | (SEQ ID No:32) |
| Primer 23 | Cfc1 | 5′-tgaaaagcttcctttcttca-3′ | 5′-tgtggtgtaatcaagggtg-3′ |
| | | (SEQ ID No:33) | (SEQ ID No:34) |

### Results

### 1) The CChIP Procedure

Referring to FIG. 1A, there is shown a schematic flow diagram outlining the key steps involved with the method according to the invention (i.e. CChIP).

The method according to the invention (i.e. referred to herein as the CChIP procedure) is based on the use of Drosophila SL2 cells as a source of carrier chromatin. SL2 cells and a small number of mammalian cells are mixed prior to preparation of nuclei and chromatin. Native chromatin fragments (1-5 nucleosomes in size) are immunoprecipitated with antisera to modified histones to yield Bound (B) and UnBound (UB) chromatin fractions that are enriched and depleted, respectively, in modified nucleosomes (as shown in FIG. 1A). The quantity and quality of precipitated chromatin (derived predominantly from Drosophila cells) was routinely checked by agarose gel electrophoresis (as shown in FIG. 1B). The inventors chose to detect mammalian DNA fragments by hot PCR (using [³²P]α dCTP), electrophoresis and PhosphorImaging, a technique in which the specificity of amplification is monitored for each assay by measuring the size of the DNA fragment generated.

It can be seen that all primer pairs employed yielded a single band of the expected size and did not cross-react with Drosophila DNA (as shown in FIG. 1C). The inventors believe that Real-Time Quantitative PCR(RTQ-PCR) could also be used to assay precipitated DNA. However, RTQ-PCR would not provide the additional specificity check of the electrophoresis step. In initial experiments, the inventors supplemented SL2 cells with 1000 or 10,000 mouse ES cells and assayed acetylation (H4K8ac) of various promoter regions. Typical results for promoter regions of Nanog (a gene active in pluripotent cells) and Gapdh (a housekeeping gene), are shown in FIG. 1D. While the Gapdh promoter showed a moderate degree of acetylation (B/UB ratios of about 1), the Nanog promoter was highly acetylated (B/UB ratio>2). The same results were achieved by standard NChIP with chromatin from 5×10⁷ cells (as shown in FIG. 1D). The enrichment of Nanog in the Bound (acetylated) fraction after CChIP was apparent over a wide range of PCR cycle numbers (not shown) and for the quantitative analyses presented here, B/UB values were calculated from duplicate PCR reactions, each carried out over two different cycle numbers (usually 38 and 41).

To validate the CChIP procedure, the inventors compared B/UB ratios determined by standard NChIP (5×10⁷ mESC) and by CChIP (1000 mESC) for 5 different histone modifications at 7 different gene regions. As shown in FIG. 1E, there is a close correlation between the values generated by the two techniques (r=0.743, p<<0.01).

### 2) Differentiation-Related Changes in Histone Modifications Across Nanog

In various model systems, H4 acetylation and H3K4 tri-methylation are consistent marks of transcriptionally active genes, while silent genes are marked by increased levels of H3K9 di-methylation (Nat. Cell Biol. 6, 73-77 (2003), EMBO J. 7, 1395-1402 (1988), Genes Dev. 18, 1263-1271 (2004), J. Cell Sci. 116, 2117-2124 (2003)). To ask whether these marks are also involved in controlling key regulator genes in ES cells, the inventors used conventional NChIP to assay their distribution across the Nanog gene in undifferentiated cells (in which Nanog is active) and cells differentiated for 7 days in culture (in which Nanog is silent). In undifferentiated cells, regions adjacent to the promoter (as shown in A-C in FIG. 2) are marked by high levels of H4K16ac, H3K4me3 and also, unexpectedly, by high levels of H3 mono-methylated at lysine 4 (H3K4me1). The promoter showed moderate or low levels of both H3K4me2 and the silencing mark H3K9me2 while downstream region F had low to moderate levels of all marks (as shown in FIG. 2).

In differentiated cells, high levels of H4K16ac, H3K4me3 and H3K4me1 at the promoter were lost while H3K9 methylation increased several fold, not only at promoter regions A-C, but also at downstream region F (FIG. 2). Changes in the silent Cdx2 gene and the housekeeping gene Gapdh were much more modest, sometimes in the opposite direction to changes in Nanog (eg. FIG. 2A), and may reflect differences in the efficiency of immunoprecipitation between cell preparations. The results presented have not been normalised to Gapdh, or any other gene, as this requires arbitrary assumptions about change or constancy of epigenetic marks during development.

### 3) Epigenetic Marking of Regulator Genes in the Blastocyst

The inventors asked whether the CChIP procedure could be used to assay histone modifications associated with specific genes in the small number of cells obtainable from one, or a few, early blastocysts. At the hatching stage, the mouse blastocyst in culture comprises about 50 cells, of which about ⅔ are trophoblasts and the rest ICM. The inventors prepared trophectoderm free of ICM by manual dissection of blastocysts (as shown in FIG. 3A) and ICM, free of trophectoderm, by immunosurgery. Three ICM and three trophectoderm preparations were separately combined and dissociated by dispase digestion. The single cell suspensions, each of no more than 100 cells, were added to 5×10⁷ SL2 cells as carrier and immunoprecipitated.

Typical results are shown in FIG. 3B. These are all from one pair of ICM/trophectoderm preparations, but all experiments shown have been done at least twice, with consistent results. The promoters of genes active in ICM, Nanog and Oct4, both showed high levels of H4 acetylation, moderate levels of H3K4me3 and low levels of H3K9me2, a silencing mark. In trophectoderm, where both genes are silent, the situation was reversed, with high levels of H3K9me2 and low levels of H4 acetylation. Conversely, the Cdx2 gene, showed low H4 acetylation and high H3K9me2 in ICM, in which it is silent, and high H4 acetylation and low H3K9me2 in trophectoderm, in which it is active (as shown in FIG. 3B). The housekeeping gene Gapdh showed the same profile of histone modification between the two cell types. The relatively low level of H3K4me3 on the Gapdh promoter in ICM and trophectoderm revealed by CChIP, is also apparent in ES cells assayed by conventional NChIP (as shown in FIG. 2B).

We have used the CChIP procedure to assay epigenetic marks in cells from just a single ICM. While genes such as Nanog can be detected in the antibody-bound fractions from some such experiments (as shown in FIG. 1D), the inventors found that in other experiments they are not detectable, even by increasing the number of PCR cycles (not shown). As discussed below, while the inventors do not wish to be bound by any hypothesis, they believe that this variability may be a consequence of the small number of cells involved (10-15, depending on the yield from immunosurgery). For all subsequent experiments, the inventors used three ICMs per ChIP.

### 4) Epigenetic Comparison of ICM and Embryonic Stem Cells

To compare the epigenetic marks associated with defined genes in ICM and mouse ES cells, the inventors used CChIP to assay five different histone modifications at defined regions across Nanog and other lineage-specific genes in both cell types.

Illustrative results are shown in FIG. 4. As with above, all experiments have been repeated at least once with consistent results. Overall, the levels of acetylated H4 (H4K16ac), H3 tri-methylated at K4 (H3K4me3) and H3 di-methylated at K9 (H3K9me2) associated with such genes were each very similar between ICM and ES cells. In both ICM and ES cells, H4 acetylation is higher at the promoters of the Nanog and Oct4 genes than at their coding or 3′ regions and much higher than at the promoters of silent genes such as Nkx2.5 (as shown in FIG. 4B). H3K4me3 is more uniformly elevated across the active Nanog and Oct4 genes, with little distinction between promoter and coding regions, but is depleted at region F, downstream of Nanog, and at the promoters of the silent genes Nkx2.5 (as shown in FIG. 4C), Cdx2 and Hhex (results not shown). Conversely, H3K9me2 was depleted at promoter proximal regions of Nanog and Oct4, but modestly elevated at 3′ coding and downstream regions of Nanog and on the silent genes Nkx2.5, Cdx2 and Hhex (as shown in FIG. 4D, and not shown).

In contrast, two modifications, H3K4me2 and H3K4me1, showed a poor correspondence between ICM and ES cells. For example, in ES cells levels of H3K4me2 were higher at the promoter proximal regions of Nanog than further downstream, while in ICM, the mark was distributed across the gene. Levels of this mark were little different on Nanog than on the promoters of the silent genes Nkx2.5, Cdx2 and Hhex, and its role, if any, in transcriptional activation or silencing remains unclear (as shown in FIG. 4E). The most striking differences between ICM and ES cells were found with H3K4me1, with 5′ region A and promoter region C of Nanog showing particularly high levels in ES cells (as shown in FIG. 4F). This result is entirely consistent with those obtained in undifferentiated ES cells by conventional NChIP (as shown in FIG. 2D). The inventors also found high levels of H3K4me1 at the promoter of Nkx2.5 in ES cells, but not ICM (as shown in FIG. 4F). No such elevation was seen in two other silent genes, Cdx2 and Hhex (as shown in FIG. 4F).

These results suggest that this histone modification is not a mark of transcriptional activity per se, despite its loss from the Nanog promoter in differentiated ES cells in which the gene is silenced (as demonstrated in FIG. 2D).

In order to provide an objective and quantitative measure of the degree of epigenetic similarity between ICM and ES cells, the inventors asked whether B/UB values for specific histone modifications on six lineage specific genes, show correlation between the two cell types. Surprisingly, the inventors found that, the robust activating and silencing marks H4K16ac, H3K4me3 and H3K9me2, individually and collectively, show a strong correlation between ICM and ES cells, as shown in Table 2.

TABLE 2
Comparative epigenetic profiling of Inner Cell Mass
(ICM) and Embryonic Stem Cells (ESC) by CChIP

| | Correlation | Number | |
|---|---|---|---|
| | coeff. | of | |
| Histone Mark | (ICM v ESC) | assays | P value |
| All marks | 0.267 | 67 | <0.05 |
| H4K16ac | 0.557 | 15 | <0.05 |
| H3K4me3 | 0.424 | 15 | >0.05 |
| H3K9me2 | 0.618 | 15 | <0.05 |
| H4K16ac + H3K4me3 + H3K9me2 | 0.479 | 45 | <0.01 |
| H3K4me1 | No correlation | 11 | — |
| H3K4me2 | No correlation | 11 | — |

Levels of the histone modifications listed were assayed by the method according to the invention (CChIP) at 15 regions within the lineage-specific genes Nanog, Oct4, Cdx2, Hhex, Nkx2 and Cfc1 in ICM and ES cells. Values for specific modifications at defined gene regions in the two cell types were used to calculate the Pearson product moment correlation coefficient (r) and probability (P) values.

Hence, for these marks; for the genes tested, the inventors were surprised to find that ES cells provide a good epigenetic model of the ICM itself. In contrast, for two marks of less well-defined function, H3K4me1 and H3K4me2, there is no correlation between ES cells and the ICM (as shown in Table 2).

### Discussion

The application of the method according to the invention (i.e. the CChIP procedure) to very small numbers of cells (100 or less) is made possible because of the high efficiency of ChIP with native, unfixed chromatin (NChIP). Its success requires that losses at every stage of the procedure are kept to a minimum. Under optimum conditions, the yield of nuclei prepared from whole cells as described, is at least 80% while the yield of chromatin in the soluble fractions (S1/S2) after micrococcal nuclease digestion is routinely >90%. The immunoprecipitation step too can be surprisingly efficient with native chromatin, particularly if nuclease digestion is controlled to give chromatin rich in oligonucleosomes, which are more efficiently precipitated than mononucleosomes. Under these conditions, with high affinity antisera, depletion of the Unbound fraction in the modified histone targeted by the antibody can be almost complete.

However, the efficiency will vary between antisera and from one chromatin preparation to another and for the purposes of the following calculation, the inventors have assumed that 40% of chromatin carrying the histone modification under test will actually be recovered in the Bound fraction. Losses during handling typically lead to a measured overall recovery after the immunoprecipitation step (Bound and Unbound fractions) of close to 50%. Thus, the inventors believe that the overall recovery in a typical experiment will be 0.8×0.9×0.4×0.5=0.14. If the (mixed) input chromatin contains 40 copies (from 20 cells) of a specific chromatin fragment containing the histone modification under test, then 5-6 (ie. 40×0.14) will be recovered in the Bound fraction, and detected by PCR. The calculation shows that detection of histone modifications associated with specific genes in a single ICM, typically yielding about as few as 15 cells after immunosurgery, is theoretically possible. Surprisingly, the inventors have demonstrated herein that it can indeed be done. However, the inventors believe that at this level, even a modest reduction in overall yield may prevent detection. The inventors believe that a reduced yield may be due to technical factors, but it could also reflect the frequency with which modified nucleosomes are distributed across the target genomic region. If, for example, at any given time only one nucleosome in ten is modified, then the overall yield will be reduced accordingly.

The inventors believe that there is no theoretical reason why the method according to the invention (CChIP) should not be effectively applied to formaldehyde cross-linked chromatin (XChIP) (Trends Biochem Sci. 25: 99-104 (2000)), thereby allowing the genomic locations of non-histone proteins to be assayed in equally small cell populations. Such proteins are usually lost during nuclease digestion of native, unfixed chromatin. The efficiency of XChIP can be high when antisera to non-histone proteins are used, but could be lower with antisera to modified histones, presumably because cross-linking of the lysine-rich histone tails to adjacent DNA alters or obscures the epitopes against which antisera are directed. Yields of less than 1% are usual, which may reduce the efficacy of the use of XChIP for analysis of histone modifications in very small cell populations. Despite this, the method according to the invention should be widely applicable to epigenetic analysis of primary cell samples obtained, for example, from biopsies of normal and diseased tissue, or by flow sorting, where cell numbers have previously been limiting.

### Epigenetic Profiling of ICM and ES Cells

The inventors have successfully used the method according to the invention to define histone modifications at the promoters of key regulator genes Nanog, Oct4 and Cdx2 in cells of the ICM and trophectoderm. The results presented herein provide a direct demonstration that the promoter regions of the crucial regulator genes Nanog and Oct4 are selectively enriched in acetylated H4 (H4K16ac) in the ICM, where they are active, but depleted in acetylated H4 and enriched in H3K9me2 in the trophectoderm, where they are silent. The converse is true for the trophectodermal gene Cdx2.5, whose promoter is highly acetylated in the trophectoderm, but enriched in H3K9me2 in the ICM. Hence, the results clearly indicate that H4K16 acetylation and H3K9 di-methylation are robust epigenetic marks for active and silent genes in cells of the early embryo, as they are in other systems. The distribution of H3K4 tri-methylation is also generally consistent with its association with active chromatin. In the ICM, trophectoderm and in ES cells, levels of this modification are generally increased in the promoter regions of active genes and decreased in silent genes, but the differences are less dramatic than for H4 acetylation and enrichment is less closely confined to promoter regions. The inventors believe that this is consistent with the recent suggestion that H3K4 di- and tri-methylation in higher eukaryotes may play a role in transcriptional elongation.

Advantages of the method according to the present invention (ie. the CChIP protocol described herein) are that it generates a quantitative measure of levels of modification (the B/UB ratio) that can be used to measure similarities and differences between ICM and ES cells. Hence, by correlating levels of a specific modification at several lineage-specific genes or gene regions, it is surprisingly possible to make a quantitative assessment of the degree of similarity between the two cell types. Such epigenetic profiling complements the gene expression profiling, made possible by PCR-based assays of mRNAs in ICM, trophoblast and ES cells and, potentially, the analysis of chromatin structure at defined loci in small numbers of cells by nuclease sensitivity (Nuc. Acids Res. 27, e32 (1999)). Previous comparative analyses of epigenetic marks in the early embryo have relied on immunofluorescence microscopy with antisera to 5meC (to detect methylated DNA) and modified histones. This approach has detected striking differences between ICM and trophectoderm, and between the ICMs of cloned and normal embryos, in levels of 5meC and methylated H3K9 (Curr. Biol. 13, 1116-1121 (2003). Immunofluorescence microscopy provides a powerful approach that can define the amount and distribution of epigenetic marks' at the single cell level, but unfortunately does not allow the detailed analysis of genes and gene regions made possible by the method according to the invention (CChIP).

Surprisingly, the results presented herein show a close and significant correlation between the distributions of H4K16ac, H3K4me3 and H3K9me2 on lineage-specific genes in ICM and ES cells. As far as these well-established marks of active and silent genes are concerned, cultured ES cells provide a good epigenetic model of the ICM.

### SUMMARY

The inventors appreciated that the method of chromatin immunoprecipitation (ChIP) has been used to study histone modifications, non-histone proteins and their modifications, and DNA methylation associated with expression or silencing of genes. However, because such studies require at least one million cells, they realised that it is impossible to use chromatin immunoprecipitation using material from experimentally and clinically important tissue sources such as the early embryo itself, with a typical inner cell mass (ICM) comprising less than 60 cells. The inventors have addressed this problem by devising a novel chromatin immunoprecipitation method (referred to herein as “CChIP”) by which histone modifications, non-histone proteins and their modifications and DNA methylation can be assayed at defined genomic regions in fewer than 100 cells, and even as low as 15 cells. Hence, this is an improvement in sensitivity of at least 4 orders of magnitude over the prior art.

Furthermore, the inventors have made a quantitative comparison of the distribution of histone modifications on key regulator genes (eg. Nanog, Oct4 and Cdx2) in ICM and cultured ES cells. They have shown that histone modifications previously closely linked to transcriptional activity and silencing (ie. H4 acetylation, H3K4 tri-methylation and H3K9 di-methylation) show closely comparable distributions in the two cell populations, while two other marks of less well-defined function (H3K4 mono- and di-methylation), are different.

### EXAMPLE 2

### A Protocol for CChIP

We provide below a method for epigenetic analysis of an analyte biological sample, in which the biological sample is subjected to native unfixed chromatin immunoprecipitation (N-ChIP).

Part 1: Chromatin Preparation from Carrier (SL2) and Target Cells

### General Points

i) To allow us to monitor the yield of chromatin in later steps, and to ensure accuracy in performing PCR, we routinely label carrier (Drosophila SL2) cells overnight with ³H-thymidine to provide an accurate and sensitive marker for bulk DNA. This is not essential. As an alternative method for determining the amount of DNA in the unbound and bound samples following immunoprecipitation we also use a pico-green assay (Molecular probes) which is also very sensitive.
ii). ALL solutions must be ICE COLD so place on ice before harvesting cells. All solutions containing sucrose must be MADE UP FRESH on the day.
iii) Remember to add protease inhibitors (whichever your lab uses) to all solutions JUST BEFORE USE. We use 0.1M PMSF and Complete mini protease inhibitors (Roche).

### Cells and Harvesting

1. Harvest SL2 cells (Note A) by spinning down at 200×g for 7 min. and wash×3 in 40 mls of ice cold PBS+5 mM Na butyrate (Note B). To achieve a consistent single cell suspension, remember to resuspend the cell pellet first by flicking the base of the centrifuge tube before adding further solutions. Count cells and divide into aliquots of 5×10⁷ cells transfer to 1.5 ml eppendorfs.
2. To each aliquot of SL2 cells (5×10⁷) add your chosen “target” cells. (Note C)
3. Spin down as above and wash pellet twice in ice cold 1 ml NB buffer+5 mM Na Butyrate.
4. Resuspend cell pellet in 2 ml NB buffer. Transfer to a 7 ml bijou (polyethylene) and add an equal volume of NB/1% Tween 40. Add a small plastic magnetic “flea” and stir gently on ice for 1 hr.
5. Homogenise sample using a hand operated, Dounce all-glass homogeniser with a “tight” pestle. After 10 smooth strokes place the homogeniser on ice until the suspension clears (usually 2-4 minutes). During the cooling/settling period, release of nuclei should be checked by examining a small aliquot under the microscope in a standard counting chamber. Whole cells and nuclei can easily be distinguished at ×40 magnification. Ideally 75-85% of cells should yield intact nuclei and this usually requires four cycles of homogenisation/cooling (Note D).
6. Following homogenisation transfer nuclei to 50 ml centrifuge tubes and spin down nuclei at 800×g for 15 minutes at 4° C.
7. Resuspend nuclei in 20 mls of 5% sucrose/NB and wash once in the same volume (800×g 15 minutes at 4° C.). (Remember to resuspend the pellet first by flicking the centrifuge tube before adding any solutions).
8. Resuspend nuclei in 5 ml Digestion buffer. Check the amount of chromatin by measuring the A₂₆₀ of an aliquot diluted 20-fold in 0.1% SDS, eg. 20 μl of Sample+380 μl 0.1% SDS.
9. An A₂₆₀ reading of 1 (in a cuvette with a 1 cm light-path) corresponds to about 50 μg/ml of chromatin DNA. So the yield of chromatin (in μg) is given by A₂₆₀× dilution factor×volume×50.
10. Centrifuge sample (800×g for 10 minutes at 4° C.) and resuspend pellet to a chromatin DNA concentration of 0.5 mg/ml. Divide into 1 ml aliquots in Eppendorf tubes (Note E).
11. Digest chromatin with micrococcal nuclease. We use 50 Upper 250 μg chromatin for 5 minutes at 28° C. Stop the digestion by addition of 0.5M EDTA to a final concentration of 5 mM and place on ICE for 5 minutes (Note F).
12. Centrifuge 12000×g for 5 minutes, remove supernatant (S1) and keep on ice.
13. Resuspend and combine pellets (if multiple micrococcal nuclease digestions have been performed) in 1 ml final volume of Lysis buffer (fraction S2). Dialyse S1 and S2 overnight at 4° C. against 2 litres of Lysis buffer (Note G).
14. Centrifuge dialysed chromatin at 1800×g for 10 minutes at 4° C. Remove supernatants and place on ice (fractions S1 and S2).
15. Resuspend pellet resulting from centrifugation of S2 in 250 μl Lysis buffer (fraction P1).

### Chromatin Analysis (Fractions S1, S2, P)

16. Check A₂₆₀ and ³H-Thymidine dpm of all samples. Calculate distribution of chromatin between the three fractions.
17. Analyse all samples by 1.2% agarose gel electrophoresis (AGE).

### DO NOT PLACE ETHIDIUM BROMIDE IN THE AGAROSE OR IN THE ELECTROPHORESIS BUFFER. DUE TO THE PRESENCE OF SDS IN THE SAMPLES THE GEL MUST BE STAINED AFTERWARDS.

18. Preparation of samples for AGE:

x μl Chromatin fraction (to give 2 μg DNA)

y μl ddH₂O (x+y=25 μl)

3 μl 1% SDS (final concentration 0.1%)

2 μl Loading buffer

19. Combine S1 and S2 fractions. Check concentration and then perform N-ChIP.

### Solutions Required for Chromatin Preparation

NB buffer [Make up 500 ml store at 4° C.]
15 mM Tris-HCl (pH7.4), 60 mM KCl, 15 mM NaCl, 5 mM MgCl₂, 0.1 mM EGTA, 0.5 mM 2 mercaptoethanol, 0.1 mM PMSF
   NB/1% Tween 40 [10 ml]
   Ensure that the Tween 40 is completely dissolved. We normally warm under the tap and keep at room temperature. If kept on ice for prolonged periods the Tween 40 comes out of solution as a white precipitate.
   25% Sucrose/NB [100 ml]
   Dissolve 25 g sucrose in NB buffer and make final volume up to 100 ml.
   Digestion buffer. [100 ml]

### 0.32 M Sucrose, 50 mM Tris-HCl (pH7.4), 4 mM MgCl₂, 1 mM CaCl₂, 0.1 mM PMSF

Lysis Buffer [2 litres]
2 mM Tris-HCl (pH7.4), 0.2 mM EDTA, 5 mM Na butyrate, 0.2 mM PMSF, 0.4 M Glycine
Plus additional protease inhibitors (Complete mini, Roche, or your equivalent).

### Part 2: Precipitation of Chromatin (NChIP)

1. Add 50-200 μl affinity-purified antibody (50-100 μg immunoglobulin) to 100-200 μg unfixed chromatin and add incubation buffer (50 mM NaCl, 20 mM Tris-HCl, pH 7.5, 20 mM Na butyrate, 5 mM Na₂EDTA, 0.1 mM PMSF) to a final volume of 1 ml. [Note I]
2. After overnight incubation (on a very slowly rotating platform) at 4° C., add 200 μl pre-swollen protein A-Sepharose (50% v/v slurry, Pharmacia) and continue the incubation for a further 3 h at room temperature. [Note J]
3. Centrifuge the antibody-chromatin mixture at 11,600 g for 10 min. Carefully remove and keep the supernatant on ice. This is the unbound fraction and should be depleted in the target protein.
4. Resuspend the protein A-Sepharose pellet in 1 ml buffer A (50 mM Tris-HCl, pH 7.5, 10 mM EDTA, 5 mM Na butyrate) containing 50 mM NaCl and layer onto 9 ml of the same buffer.
5. After centrifugation at 1200 rpm, for 10 min at 4° C., remove the supernatant by aspiration and wash the pellet in 10 ml buffer A containing 100 mM NaCl and finally in 10 ml of buffer A containing 150 mM NaCl. [Note K]
6. Elute the bound material from the protein A-Sepharose by addition of 125 μl 1% SDS in incubation buffer and incubate for 15 min at room temperature with repeated inversion. After centrifugation at 11,600 g for 10 min remove and KEEP the supernatant (bound 1) and store on ice.
7. Repeat this step and combine the supernatant with bound 1 to give the final bound fraction. Add an equal volume of incubation buffer to the bound fraction to reduce the concentration of SDS to 0.5%.

### Isolation of DNA

8. Add one-third volume of phenol:chloroform (1:1) to the input, unbound, and bound fractions.
9. Vortex and centrifuge at 600 g for 10 min at 4° C. to separate the phases.
10. Remove the supernatant and add an equal volume of phenol:chloroform. Repeat the centrifugation.
11. Add an equal volume of chloroform, centrifuge as before, and transfer the supernatant to a 6-ml centrifuge tube.
12. Finally precipitate the DNA at −20° C. using 1/100th vol of 4 M LiCl, 25 μl glycogen (2 mg/ml) and 2 volumes of ice-cold ethanol. [Note L]

### Analysis of DNA Following NChIP

13. Check ³H-Thymidine dpm of all samples. Calculate percentage pull-down for each antibody. Compare to the preimmune control. [Note L]
14. Analyse all samples by 1.2% agarose gel electrophoresis (AGE) to check if pull down has worked. [Note L]
15. Perform Species specific PCR on equal amounts of DNA from the unbound and bound samples [Note M]

### Isolation of Proteins

16. Precipitate the proteins from the first phenol:chloroform phase by addition of 5 μg BSA (carrier), 1/100th vol 10 M H₂SO₄, and 12 volumes of acetone.
17. After overnight precipitation at −20° C. wash the protein pellets once in acidified acetone (1:6 100 mM H₂SO₄:acetone) and three times in dry acetone. [Note N]

### Solutions Required for Immunoprecipitation Experiments

### Incubation Buffer

50 mM NaCl, 20 mM Tris-HCL (pH 7.5),

20 mM Na butyrate, 5 mM Na₂EDTA,

0.1 mM PMSF

### Buffer A

50 mM Tris-HCL (pH 7.5), 10 mM EDTA

50 mM NaCl, 5 mM Na butyrate

### Buffer B

500 mM Tris-HCL (pH 7.5), 10 mM EDTA,

100 mM NaCl, 5 mM Na butyrate

### Buffer C

50 mM Tris-HCL (pH 7.5), 10 mM EDTA,

150 mM NaCl, 5 mM Na butyrate

### Protein A Sepharose (Pharmacia)

Pre-swell protein A sepharose overnight in buffer A at 4° C.

After centrifugation (2000 rpm 10 minutes MSE Chilspin)

resuspend pellet in an equal volume (50% w/v) of Buffer A

### Notes on the CChIP Protocol

### Note A

Drosophila SL2 cells are grown at 26° C. in Schneider's medium (Gibco) supplemented with 8% foetal calf serum (Gibco) and antibiotics (50 units per ml Penicillin, 50 μg per ml Streptomycin). We usually grow these cells in standard 75 cm² tissue culture flasks laid flat and with sealed caps (gassing is unnecessary). The cells grow in suspension as loose clumps and are usually split 1 to 4 every two weeks. Pellets of SL2 cells (ideally 5×10⁷ cells) can be safely stored at −80° C. for two months or more prior to use in CChIP. But if you use frozen SL2 cells, then use only one round of homogenisation (ie. 10 strokes) to release the nuclei (step 4 below). Check the nuclei: cell ratio and if necessary give 5 more strokes. The frozen nuclei are more fragile than fresh ones and more easily lysed.

### Note B

We have added Na butyrate to prevent deacetylation during the isolation procedure and this is present in all our solutions, along with protease inhibitors.

### Note C

This will usually be between 100 and 10,000 cells. The minimum number of cells on which we have successfully performed CChIP is 50-100, BUT at such low cell numbers, the number of PCR reactions that can be performed on Bound and Unbound fractions is inevitably small. Detection of target cell DNA requires a very high efficiency of immunoprecipitation and minimal experimental losses.

### Note D

It is important that steps 4 and 5 are carried out promptly and that cell lysis and release of nuclei is efficient. The presence of large numbers (>20%) of intact cells has a detrimental effect on the quality of the chromatin after micrococcal nuclease digestion.

### Note E

We have found that a more reproducible digestion is achieved if the micrococcal nuclease step is performed in 1 ml aliquots rather than larger volumes.

### Note F

This step MUST be carefully timed and controlled. Over-digestion will lead to sub-nucleosomal particles and possible degradation of target sequences. Under-digestion will lead to a reduced overall yield of chromatin and possible under-representation of chromatin from more condensed regions of the genome. Note that the NChIP procedure has consistently given preferential precipitation of oligonucleosomes in comparison to mononucleosomes. For efficient precipitation, a chromatin preparation consisting predominantly of oligonucleosmes (centered around 3-5mers) is ideal.

### Note G

We have found that it is not necessary to dialyse S1 at this point. However, if S1 is not dialysed, be sure to add glycine to S1 at a final concentration of 0.4M to make it equivalent to the S2 fraction. If this is not carried out then S1, S2 and P fractions will electophorese differently on agarose gels.

### Note H

If after combining S1 and S2 the sample is too dilute to achieve the concentration required for ChIP (ideally 200 μg/ml) then concentrate the sample using a Centricon concentrator.

### Note I

We have found that the use of affinity-purified antibodies reduces the amount of nonspecific binding. The optimum amount of antibody added is dependent on its titer and on the amount of target protein present in the chromatin and must be determined for each antiserum. Commercially available antiserum has been employed successfully in XChIP using very small amounts (5-50 μl) and subsequent analysis by PCR. We always use affinity purified antisera for ChIP and either whole sera or 50% saturated ammonium sulphate cuts for Western blotting.

### Note J

Protein A-Sepharose is available commercially as a freeze-dried powder and should be preswollen in 50 mM Tris-HCl, 5 mM Na EDTA, 50 mM NaCl. The concentration of the NaCl can be increased depending on the affinity of the antibody for its target protein. Increasing the NaCl concentration reduces the amount of nonspecific binding, but may also reduce the binding affinity of the antibody for the target protein. Preliminary experiments changing the NaCl concentration should be employed to determine the optimum conditions for the antibody used.

### Note K

The concentration of the NaCl can be increased to reduce nonspecific binding. We have found that large volume washes, carried out in 15-ml siliconized centrifuge tubes, greatly reduce nonspecific binding.

### Note L

We routinely add glycogen (5 μg) as a carrier to maximize the precipitation of DNA from the bound fraction. Ethanol used in the precipitation should be molecular biology grade (e.g., Analar-BDH) and stored at −20° C. to ensure rapid precipitation.
The volume you re-suspend your pellet in following immunoprecipitation is VERY IMPORTANT in CChIP. Remember that both the Unbound and Bound samples will have low amounts of ‘target’ DNA in them. If you have spiked with a very low number of cells (100 say) then remember that you will have only 200 molecules of any particular DNA fragment. With an overall yield of 40%, then you will have 80 molecules in the Bound and Unbound fractions combined. Decide how many fragments do you need per PCR reaction and resuspend accordingly. The total amount of DNA in each fraction is determined by the amount of [³H] thymidine present (using scintillation counting) and one must assume that the recovery for the ‘carrier’ and target will be equivalent.
Remember to keep the first phenol:chloroform phase for subsequent protein isolation. We routinely analyze the DNA samples by electrophpresis on 1.2% agarose gels followed by staining with ethidium bromide.

### Note M

In order to detect your ‘target’ DNA species-specific PCR must be performed. All ‘target’ primers should be checked for cross reactivity against ‘carrier DNA’, both by BLAST search and by visualisation following test PCR. We have found that PCR primer sets that should be mouse-specific according to the BLAST search, can still cross react with Drosophila when tested by radioactive PCR, normally giving a higher molecular weight product. We have chosen to use radioactive PCR followed by analysis on DNA PAGE, drying down of gels and exposure to phosphorimager screens for our routine analyses. Initial analyses establish the linear range for each primer set. This has proved a reliable and quantitative method that confirms both that the assayed product is of the right size and absence of cross-reactivity with the ‘carrier’ DNA. Alternatively, analysis of samples could be performed by Real Time Quantitative-PCR, though we would recommend use of Taqman probes for the added specificity they provide.

### Note N

We routinely analyze the proteins by electrophoresis on SDS-polyacrylamide gels. If proteins are to be analyzed on acid/urea/Triton (AUT) gels, the acetone pellet should be resuspended in 500 μl double-distilled H₂O and centrifuged through microconcentrators (Amicon) for 30 min at 11,600×g to reduce the volume. This dilution:concentration step is repeated (to reduce residual SDS) and 2 vol of AUT loading buffer (8 M urea, 5% 2-mercaptoethanol, 1 M glacial acetic acid, plus a few drops of tracking dye (pyronine Y)) is added to the final concentrated sample. Western blotting and immunostaining are carried out using standard methodology.

### REFERENCES

O'Neill L P & Turner B M (2003) Methods 31(1) 76-82
O'Neill L P, VerMilyea M D, Turner B M (2006) Nature Genetics 38 (7) 835-841

### EXAMPLE 3

### X-ChIP Protocol for Use with CChIP

In Example 2 above, the method for epigenetic analysis of the invention uses the native unfixed chromatin (NChIP) procedure on an analyte biological sample. However, as discussed further above, the method for epigenetic analysis can also employ the X-ChIP procedure. We provide below an X-ChIP protocol that can be used in accordance of the method of the invention. Moreover, X-ChIP is a well known laboratory procedure and laboratory protocols can be readily identified and adapted for use in the method of the invention.

As a first step, the analyte biological sample (e.g. analyte cells) are mixed with carrier chromatin, such as Drosophila SL2 cells as set out above in Example 2. The mixture is then resuspended in a “fixation buffer” (100 mM NaCl, 1 mM EDTA, 1 mM EGTA, 50 mM HEPES pH 8, 1% paraformaldehyde) for 8 min at 37° C. The amount of formaldehyde used in the fixation buffer can be varied so as to determine the quantity required to provide optimal crosslinking of proteins to chromatin. The mixture is then washed twice in ice cold PBS (a standard molecular biology reagent) and resuspended in cell lysis buffer (5 mM PIPES, 85 mM KCl, 0.5% Nonidet P40, imM PMSF+protease inhibitors) at a concentration of 4×10⁷ cells/ml.

After 10 min incubation on ice, nuclei are prepared by Dounce homogenisation (as in the standard procedure), spun down and resuspended in nuclear lysis buffer (50 mM Tris-HCl pH 8.1, 10 mM EDTA, 1% SDS, 1 mM PMSF, 5 mM Na butyrate, +protease inhibitors) at a concentration of 1×10⁸ cells/ml. After stirring on ice for 10 min, the extracts are sonicated to fragment the DNA. This can be done using a Diagenode BioRuptor sonicating water bath designed specifically for preparing material for XChIP.

At this point the protocol follows the procedure set out in Example 2 above, with the exception that the precipitated/unprecipitated chromatin samples should be heated at 65° C. overnight to reverse cross links before DNA purification and PCR analysis.

### EXAMPLE 4

### Preparation of Different Analyte Samples

In the examples above, the cells used as analyte samples are mouse ICM and cultured ES cells. However, the method for epigenetic analysis of the invention can be applied to many different analyte populations.

For example, the inventors have used fluorescent-activated cell sorting (FACS) to sort 10,000 mouse embryo fibroblasts in to populations based on position in the cell cycle (i.e. cells are sorted according to whether they are in G1, S, G2 or M stage of the cell cycle). The dye used for the FACS was Vybrant DyeCycle (Invitrogen), which is a cell-permeable dye that binds to double stranded DNA. However, as would be appreciated, other populations of cells sorted by FACS according to cell surface antigens can readily be used in the method for epigenetic analysis of the invention. Following FACS, 5000 sorted cells were then used in the method for epigenetic analysis of the invention which employed antibodies to H4 acetylated at lysine 8 (H4K8ac) and H3 di-methylated at lysine 4 (H3K4me2).

Alternatively, the method of the invention can be applied to analyte samples taken from tumour biopsy material. For example, frozen sections of cancer tissue (e.g cervical cancer, Hodgkin's lymphoma) fixed in ethanol can be used to prepare chromatin suitable for use in the N-ChIP protocol. Accordingly, such sections can be used as analyte samples in the method of the invention.

1. A method of carrying out epigenetic analysis on an analyte biological sample, the method comprising carrying out chromatin immunoprecipitation on an analyte biological sample, characterised in that the method comprises a step of contacting the analyte biological sample with carrier chromatin before the step of carrying out chromatin immunoprecipitation on the analyte biological sample.

2. The method of claim 1 further comprising analysing or detecting one or more epigenetic marks in the analyte biological sample.

3. The method of claim 2 wherein the epigenetic mark is histone protein modification, non-histone protein modification and/or DNA methylation.

4. The method of claim 1 wherein the analyte biological sample comprises less than one million cells.

5. The method of claim 1 wherein the analyte biological sample comprises mammalian cells.

6. The method of claim 5 wherein the cells are human or mouse cells.

7. The method of claim 1 wherein the analyte biological sample comprises less than about 6 μg of DNA and/or less than about 12 μg of chromatin.

8. (canceled)

9. The method of claim 1 wherein the step of contacting the analyte biological sample with carrier chromatin comprises mixing carrier chromatin with chromatin derived from the analyte biological sample.

10. The method of claim 1 wherein the amount of carrier chromatin is at least 10 times the amount of chromatin in the analyte biological sample.

11. The method of claim 1 wherein the DNA of the chromatin of the analyte biological sample is distinguishable from the DNA of the carrier chromatin.

12. The method of claim 1 wherein where the analyte biological sample is not derived from Drosophila then the carrier chromatin is derived from Drosophila cells.

13. The method of claim 12 wherein the Drosophila cells are SL2 cells.

14. The method of claim 1 wherein the chromatin immunoprecipitation is N-ChIP.

15. The method of claim 1 wherein the chromatin immunoprecipitation is X-ChIP.

16. The method of claim 1 comprising the steps: (I) mixing carrier chromatin with analyte biological sample comprising cells; (II) disrupting the cells in step (I) to release nuclei therefrom; (III) digesting the nuclei to release the chromatin therefrom; (IV) immunoprecipitating the chromatin obtained from step (III) using an antibody specific to a protein of interest; (V) subsequently purifying DNA from the isolated protein/DNA fraction; (VI) analysing DNA fragments isolated in connection with the protein of interest.

17. A method of identifying histone protein modification, non-histone protein modification, and/or DNA methylation, or patterns thereof, in an analyte biological sample, the method comprising carrying out chromatin immunoprecipitation on an analyte biological sample, characterised in that the method comprises a step of contacting the analyte biological sample with carrier chromatin before the step of carrying out chromatin immunoprecipitation on the analyte biological sample.

18. A method for aiding the diagnosis or prognosis of a disease condition comprising performing the method of claim 1 or claim 17.

19. The method of claim 18 wherein the disease is cancer or an autoimmune disease.

20. The method of claim 18 wherein the method comprises performing comparative epigenetic analysis, or comparative investigations of histone protein modification, non-histone protein modification, and/or DNA methylation, or patterns thereof, of a normal analyte biological sample to an analyte biological sample comprising tissue or cells under investigation.

21. The method of claim 20 wherein the analyte biological samples comprise cells from tumourous and/or non-tumourous tissues.

22. The method of claim 20 wherein the analyte biological samples comprise cells from formaldehyde-fixed or ethanol-fixed tissue section.

23. A method of carrying out epigenetic analysis on a sample of stem cells or a stem cell precursor, the method comprising carrying out chromatin immunoprecipitation on a sample of stem cells or a stem cell precursor, characterised in that the method comprises a step of contacting the stem cells or stem cell precursor with carrier chromatin before the step of carrying out chromatin immunoprecipitation on the sample of stem cells or stem cell precusor.

24. The method of claim 23 wherein the stem cell precursor comprises one or more cells from the ICM, such as the trophectoderm or trophoplast.

25. The method of any claim 23 wherein the epigenetic analysis comprises analysis of one or more regulator genes in the stem cells or precursors.

26. The method of claim 25 wherein the regulator genes analysed are independently selected from Nanog, Oct4, and Cdx2.

27. An epigenetic analysis kit comprising one or more materials for performing chromatin immunoprecipitation, characterised in that the kit further comprises carrier chromatin.

28. The kit of claim 27 wherein the carrier chromatin comprises Drosophila SL2 cells.


SEQUENCE LISTING (34 sequences)

SEQ ID NO: 1 - DNA, 19 bp, Artificial
  Synthetic primer
gtaaagcctctttttgggg

SEQ ID NO: 2 - DNA, 17 bp, Artificial
  Synthetic primer
caccagccctgtgaatt

SEQ ID NO: 3 - DNA, 19 bp, Artificial
  Synthetic primer
ctatcgccttgagccgttg

SEQ ID NO: 4 - DNA, 26 bp, Artificial
  Synthetic primer
aactcagtgtctagaaggaaagatca

SEQ ID NO: 5 - DNA, 20 bp, Artificial
  Synthetic primer
tcacactgacatgagtgtgg

SEQ ID NO: 6 - DNA, 20 bp, Artificial
  Synthetic primer
tctgtgcagagcatctcagt

SEQ ID NO: 7 - DNA, 20 bp, Artificial
  Synthetic primer
gtggagcgctaggaagtgtg

SEQ ID NO: 8 - DNA, 20 bp, Artificial
  Synthetic primer
agccagggctacacagagaa

SEQ ID NO: 9 - DNA, 20 bp, Artificial
  Synthetic primer
gaagacctgcctcttcaagg

SEQ ID NO: 10 - DNA, 19 bp, Artificial
  Synthetic primer
agaacacagtccgcatctt

SEQ ID NO: 11 - DNA, 20 bp, Artificial
  Synthetic primer
accagcccctggtttatttt

SEQ ID NO: 12 - DNA, 20 bp, Artificial
  Synthetic primer
ggcaaagataagtgggcaga

SEQ ID NO: 13 - DNA, 17 bp, Artificial
  Synthetic primer
aaatcgtgtttctgggg

SEQ ID NO: 14 - DNA, 20 bp, Artificial
  Synthetic primer
ccttacgtgattaacgagtg

SEQ ID NO: 15 - DNA, 18 bp, Artificial
  Synthetic primer
tagggtcagcacttccag

SEQ ID NO: 16 - DNA, 20 bp, Artificial
  Synthetic primer
cacagtcttgcaagatgaag

SEQ ID NO: 17 - DNA, 21 bp, Artificial
  Synthetic primer
tgtgccaagcacttgtataac

SEQ ID NO: 18 - DNA, 22 bp, Artificial
  Synthetic primer
tatgtctgaccagaggagagca

SEQ ID NO: 19 - DNA, 18 bp, Artificial
  Synthetic primer
tcccccgttctagacagt

SEQ ID NO: 20 - DNA, 17 bp, Artificial
  Synthetic primer
agcctctggaacctgga

SEQ ID NO: 21 - DNA, 20 bp, Artificial
  Synthetic primer
ctgtaaggacaggccgagag

SEQ ID NO: 22 - DNA, 20 bp, Artificial
  Synthetic primer
caggaggccttcattttcaa

SEQ ID NO: 23 - DNA, 20 bp, Artificial
  Synthetic primer
ggatggcatactgtggacct

SEQ ID NO: 24 - DNA, 20 bp, Artificial
  Synthetic primer
agttgctttccactcgtgct

SEQ ID NO: 25 - DNA, 20 bp, Artificial
  Synthetic primer
caaggcaagggaggtagaca

SEQ ID NO: 26 - DNA, 20 bp, Artificial
  Synthetic primer
gctcctgatcaacagcatca

SEQ ID NO: 27 - DNA, 18 bp, Artificial
  Synthetic primer
gatttcacacccaccctc

SEQ ID NO: 28 - DNA, 18 bp, Artificial
  Synthetic primer
ccggtcctagtgtggaat

SEQ ID NO: 29 - DNA, 20 bp, Artificial
  Synthetic primer
caaagctctttggtttgttg

SEQ ID NO: 30 - DNA, 20 bp, Artificial
  Synthetic primer
agcctcctggtgctatttac

SEQ ID NO: 31 - DNA, 18 bp, Artificial
  Synthetic primer
tagggtcagcacttccag

SEQ ID NO: 32 - DNA, 20 bp, Artificial
  Synthetic primer
cacagtcttgcaagatgaag

SEQ ID NO: 33 - DNA, 20 bp, Artificial
  Synthetic primer
tgaaaagcttcctttcttca

SEQ ID NO: 34 - DNA, 19 bp, Artificial
  Synthetic primer
tgtggtgtaatcaagggtg